(19) United States
(12) Patent Application Publication
(10) Pub. No.: US 2010/0036369 A1
(43) Pub. Date: Feb. 11, 2010

(54) MICROWAVE ARRAY APPLICATOR FOR HYPERTHERMIA

(75) Inventor: Christopher Paul Hancock, Avon (GB)

Correspondence Address: ARENT FOX LLP, 1050 CONNECTICUT AVENUE, N.W., SUITE 400, WASHINGTON, DC, 20036, US

(73) Assignee: Bangor University, Gwynedd (GB)

(21) Appl. No.: 12/518,210

(22) PCT Filed: Dec. 5, 2007
(86) PCT No.: PCT/GB2007/004660

§ 371 Date: Oct. 23, 2009

(30) Foreign Application Priority Data
Dec. 8, 2006 (GB) 0624584.9

(51) Int. Cl. A61B 18/18

(52) U.S. Cl. 606/33

(57) ABSTRACT

Apparatus (10) for treating skin tissue with microwave radiation (e.g. having a frequency of 1 GHz to 300 GHz) is disclosed in which an array of radiating elements (18), e.g. patch antennas are arranged on a flexible treating surface (16) for locating over and conforming with a region of skin tissue (24) to be treated. The radiating elements (18) receive microwave energy from a feed structure and are configured to emit outwardly a electromagnetic field which permits the region of skin to a substantially uniform penetration depth. Each radiating element (18) may have an independently controllable power supply to permit relative adjustment of the field across the treatment surface. Each radiating element may have a monitoring unit to allow adjust based on detected reflected power. Each independently controllable power supply may include a dynamic impedance matching unit.

### FIELD OF THE INVENTION

This invention relates to apparatus for and methods of producing controlled thermal energy in the treatment of tissue using microwave techniques. It particularly relates to the controlled use of thermal ablation (e.g. causing tissue necrosis) as a means for treating dermatological conditions.

### BACKGROUND TO THE INVENTION

Skin is the largest organ in the human anatomy and it covers the complete surface of the body. A wide variety of skin diseases and disorders, including skin cancer are known for which direct treatment of the skin tissue itself is required to alleviate or cure symptoms. Moreover, methods of treating skin for cosmetic purposes, e.g. tissue resurfacing or skin rejuvenation are becoming increasingly common. Conventional skin treatment techniques include: laser therapy, photodynamic therapy, cryosurgery, mechanical dermabrasion, and plasma resurfacing.

Skin cancer is the most common form of cancer, and conventional treatment methods tend to be somewhat limited. Many types of skin lesions resemble common moles, which get larger and expand into the deeper layers of the skin; upon reaching the dermis, cancerous cells can enter the blood vessels and spread, or metastasize, to other parts of the body. The stage of the cancer indicates the extent of the disease and is determined by the depth that the lesion penetrates into the skin, and by how much it has spread. One example of how stages of growth may be defined is as follows:

Stage 0—the cancer is in the epidermis and has not begun to spread.

Stage 1—localised tumour that is 0.75 mm or less in thickness and has spread to the upper dermis.

Stage 2—localised tumour that is thicker than 0.75 mm but less than 1.5 mm and/or begins to invade the lower dermis.

Stage 3—localised tumour that is more than 1.5 mm but not more than 3 mm in thickness.

Stage 4—localised tumour that is thicker than 1.5 mm but less than 4 mm and/or invades the lower dermis.

Stage 5—localised tumour that is greater than 4 mm in thickness and/or invades the subcutaneous tissue (tissue beneath the skin) and/or satellites within 2 cm of the primary tumour.

Stage 6—the tumour has spread to nearby lymph nodes or less than five in-transit metastases are found. An in-transit metastasis is a metastasis that is located between the primary tumour and the closest lymph node region and results from melanoma cells getting trapped in the lymphatic channels.

Stage 7—the tumour has metastasised to other parts of the body.

Known skin treatment systems are inflexible because they are unable to operate on all of the different stages of skin cancer. The term “skin cancer” is a very broad due to the fact that there are several kinds of skin tumours from benign to malignant. The diagnosis of melanoma should be carried out carefully in accordance with the ABCD(E) criteria.

Other skin treatment techniques include controlled ‘sealing’, or instant cauterisation to controlled depths of penetration to stop bleeding or fluid weeping from tissue subsequent to skin graft surgery or injury. Conventional methods of achieving these effects can cause patient discomfort (pain and irritation) and require substantial tissue healing time, as well as the need for bandaging, which may need to be replaced periodically. The conventional techniques are therefore not time or cost efficient.

To address this, U.S. Pat. No. 6,463,336 discloses a conformable bandage which incorporates a pliable planar microstrip or slotline antenna structure for treating soft tissue under the bandage with a pulsed electromagnetic field, e.g. to improve the healing of wounds or to enhance transdermal drug delivery.

### SUMMARY OF THE INVENTION

The present invention provides a clinical treatment apparatus for the treatment of skin lesions and other skin conditions.

At its most general, the present invention proposes a treatment device and method which produces and uses a non-ionising microwave electromagnetic field to penetrate skin tissue to cause controllable thermal damage to that tissue in terms of depth of penetration, and uniformity of effect over the desired treatment area.

In this specification, the term ‘microwave’ is used generally to denote a frequency range from 1 GHz to 300 GHz or more. It may include high frequencies that can be said to reside in the mm wave region. In the examples given below, however, the preferred frequency is above 10 GHz. For example, spot frequencies of 14.5 GHz, 24 GHz, 31 GHz, 45 GHz, 60 GHz, 77 GHz and 94 GHz are possible.

Preferably, the present invention provides means for producing controllable uniform thermal ablation (or cell destruction) with a depth of penetration less than 5 mm, preferably less than 2 mm. For example, it may be desirable to have a range of penetration depths from 0.1 mm to 2.0 mm.

For the purposes of explaining the invention, the skin may be considered to comprise two main layers: an upper (top) top layer called the epidermis and a lower (bottom) layer called the dermis.

Using the present invention, it may be possible to deliver microwave energy only inside the epidermis. This can be desirable since damage to the dermis may cause permanent damage to the structure of the skin, or prolong healing time. Furthermore, it may make the invention suitable for use in skin rejuvenation or resurfacing procedures, where it is highly undesirable to penetrate into the dermis.

The invention may also be used for depilation of large clusters of hair on the surface of the body, for example, on the back or legs of a human being. In this application the depth of penetration of the microwave energy may be such that the roots of the hair follicles are destroyed, which should result in permanent removal of hair.

One advantage of the controllable microwave radiation of the present invention is the ability of the system to instantaneously deliver energy to produce controlled coagulation with controllable depths of penetration of e.g. less than 5 mm (preferably less than 2 mm) and field uniformity over surface areas where treatment is required. Typically, the size of surface areas to be treated can be from less than 0.5 cm² to more than 15 cm². The treatment technique proposed may also help to reduce the possibility of bacteria entering open tissue or wounds by raising the temperature to a level where bacteria are killed.

The present invention may also help to reduce significantly patient turn-around times, reduce the cost of treatment, and shorten waiting lists. The conditions that are treatable using this invention are typically those that benefit from the ability to produce uniform, and finely controlled, thermal damage over surface areas of less than 0.5 cm² to greater than 15 cm², with depths of penetration of less than 0.4 mm to greater than 5 mm. Current conventional treatment systems are not capable of producing such treatment conditions. For example, conventional laser treatment has only a small region of effect and accurate scanning is required to treat a larger area. Furthermore, topical treatments such as antibiotic gel or cream take time to have any effect, which can be inconvenient. It may also be undesirable to introduce antibiotics into a biological system. Antibiotic treatments often begin to be ineffective when used for long periods of time and may cause the body's immune system to become less efficient.

The present invention may provide an alternative to these types of treatment.

The present invention may be put into effect using semiconductor power devices which have been developed recently for the communications industry. These devices enable energy to be generated at frequencies contained within the electromagnetic spectrum that have not previously been explored or exploited for use in biomedical treatment applications. The depth of penetration of energy from an electromagnetic field into a biological tissue load depends inter alia on the inverse of the frequency of that field. Hence, for penetration into the upper layers of skin tissue only, high microwave frequency energy sources (e.g. energy sources with frequencies above 10 GHz) are desirable.

In a first aspect, the present invention relates to a skin applicator device arranged to deliver a microwave electromagnetic field into skin tissue. According to the present invention, there may be provided a device for treating skin tissue with microwave radiation, the device having: a treating surface for locating over a region of skin to be treated; a plurality of radiating elements on the treating surface; and a feed structure arranged to deliver microwave energy to the radiating elements; wherein the radiating elements are configured to emit outwardly the delivered microwave energy as an electromagnetic field at the treating surface, such that, during treatment the emitted electromagnetic field penetrates the region of skin to be treated to a substantially uniform predetermined depth.

Preferably, the feed structure includes a plurality of power sources (e.g. power amplifiers), each power source being associated with a group of (one or more) radiating elements. The power sources are preferably in close proximity to the radiating elements. This gives the feed structure two advantages that are particularly relevant for the high operating frequencies preferred in the invention. Firstly, by performing amplification close to the radiating structure, the loss in power due to transferring high frequency microwave power along transmission lines can be reduced, i.e. the insertion loss along a suitable 50 Ω microstrip transmission line transmitting at signal at a frequency of 45 GHz may be up to 10 dB per 10 cm. Secondly, the proximity of the power sources to the radiating elements allows the feed structure between the power sources and radiating elements to be simple structures, i.e. there is no need to use power splitters or combiners that add additional complexity and insertion loss if each radiating patch or element of the antenna array has its own dedicated power device. A further advantage of using this arrangement is that that it is not necessary to drive the power device into saturation, which may reduce the level DC power dissipation or may enable the device to be operated with a higher microwave power to DC power efficiency. This enables a balance to be struck between power losses (which are higher from finer transmission structures) and control of the radiating field configuration (which enables the better uniformity of the total field to be achieved).

Preferably, each radiating element has an independently controllable power source, whereby the emitted electromagnetic field is adjustable across the treatment surface. Thus, the present invention may provide an adaptive treatment apparatus capable of adjusting for the differences in skin properties across a treatment site, whereby uniform power delivery across the skin surface of the treatment site may be achieved.

The radiating elements preferably define an antenna structure, which, together with the feed structure, may be optimised to propagate energy into representative tissue impedances. The distribution of the energy is preferably uniform in terms of depth of penetration over the treatment area.

Preferably, the microwave energy has a frequency in the super high frequency (SHF) or extremely high microwave (EHF) ranges of the electromagnetic spectrum, where the associated wavelengths, when propagated into biological tissue (e.g. various types of skin tissue), are such that controllable thermal damage is produced in the tissue. Typically, these frequency ranges are 3 to 30 GHz (SHF) and 30 to 300 GHz (EHF). Such frequencies and/or frequency sources are not used in conventional biomedical treatment applications because it has been impossible or impractical to produce controllable power at such frequencies. However, by making use of recent developments in semiconductor power technology, the present inventor has overcome some of those impracticalities.

Preferably, the microwave energy has a frequency of more than 10 GHz to enable it to be useful for treating skin structures.

The device of the present invention may improve upon conventional systems by providing precision control of the thermal damage produced in terms of depth of effect, uniformity of effect over the treating surface area, and the ability to instantly raise the temperature to a level that will destroy unhealthy tissue in applications relating to the treatment of skin lesions, or to produce surface ablation to instantly stop wound bleeding, fluid weeping, or the prevention of bacteria from entering open wounds in applications related to skin graft or accident damage treatment.

Preferably, the microwave electromagnetic field emitted by the radiating elements is arranged to heat substantially instantaneously the region of skin to be treated to a temperature of 45° C. or more, preferably 60° C. or more, e.g. 60° C. up to 100° C. Such temperatures effect permanent damage of tissue structures in the region of skin to be treated. For example, exposure of cancerous tissue to temperatures of 60° C. or greater guarantees cell death.

In certain embodiments, the plurality of radiating elements may be on an outward facing surface of a dielectric substrate layer, a grounded conductive layer can be formed on a surface of the dielectric substrate layer opposite the outward facing surface, and the feed structure is arranged to deliver an alternating current to the plurality of radiating elements, the grounded conductive layer being arranged to provide a return path for the alternating current.

In other embodiments, the grounded conductive layer may be on the outwardly facing side of the dielectric substrate layer. For example, slots may be formed in the grounded conductive layer and dielectric substrate layer opposite a microstrip feed line or a coplanar waveguide fed suspended patch antenna arrangement may be employed. For the slot antenna arrangement, the slots may then act as radiating elements. The slots may have increasing width along the length of the feed line such that the same amount of microwave energy is delivered from each radiating slot to enable a uniform field to be radiated into the tissue structure.

Preferably, each radiating element includes a conducting patch mounted on the outward facing surface of the dielectric substrate layer, e.g. as slots, radiating patches or the like. For example, miniature microstrip antennas, or millimetre wave antennas fabricated using micromachining technology may be used.

Alternatively, the radiating elements may comprise a plurality of suspended patch antennas which are fed by micro-machined coplanar waveguides. This structure may be particularly useful at frequencies in excess of 20 GHz, i.e. 24 GHz, 31 GHz, 45 GHz, 60 GHz or more (i.e. at so-called ‘millimetre’ wave frequencies).

Thus, the device may include a patch antenna array on the treating surface which is configured to produce controlled microwave radiation for treating skin tissue. The patch antenna array is preferably configured to produce uniform tissue ablation over the treating surface area with a predetermined depth of penetration commensurate with e.g. the thickness of skin tumours, other skin diseases, and wound healing.

Additionally or alternatively, the device may be used to instantly coagulate blood or blood flow, or weeping fluid subsequent to skin removal. This application is feasible because the present invention uses microwave power at very high frequencies which make it possible to achieve depths of penetration that are or interest for surface coagulation. Previously, it was difficult to produce controllable energy at high enough frequencies to ensure depths of penetration of radiation low enough to be of interest to produce controlled tissue damage with depths of penetration between less than 1 mm to around 5 mm. Higher frequency microwave energy may also ensure that chain coagulation of blood does not occur; this may be difficult when lower microwave frequencies are used due to the associated depths of penetration of the microwave energy at these lower frequencies.

A particular advantage of the invention may be the ability to reduce the amount of bacteria entering open tissue or wounds. This is achieved by the instantaneous nature of energy delivery, small depths of penetration, uniform tissue effect, the ability to treat relatively large surface areas, and the capability to produce instant heat at temperatures high enough to kill bacteria.

It is preferable to produce patches with dimensions comparable to a half the wavelength at the frequency of operation. Preferably, the area of the radiating elements is 1 mm² or less. Since the frequency is inversely proportional to the requisite half wavelength, patch dimensions of this order are achieved by using high microwave frequencies. This is due to the fact that patches with these, or similar, dimensions for width and length radiate efficiently along the edges associated with the width of said patch. In theory, the field can be zero along the length and maximal along the width. Thus, each conducting patch is preferably rectangular and configured to emit the electromagnetic field in its fundamental (TM₁₀) mode. Radiation from a single patch occurs normally from fringing fields between the periphery of the patch and the grounded conductive layer. To enable fundamental mode (TM₁₀) excitation, the length of a rectangular patch is preferably made slightly smaller than half the loaded wavelength. Other modes and suitable geometrical configurations may be used.

Alternatively, a plurality of travelling wave antenna structures placed adjacent to one another may be used.

For higher microwave frequencies, coplanar waveguide fed suspended patch antenna arrays are preferred.

The invention may be viewed as the use of high microwave (or millimetre wave) frequency energy to enable a beneficial interrelationship of three factors:
small patch size;
field uniformity over a surface of an array of patches;
depth of penetration of energy that is useful for controllably treating various structures of the skin.

When the energy propagates into skin tissue and the applicator is in contact with the skin surface, the loading comes from the relative permittivity of the dielectric substrate layer and the relative permittivity of the biological tissue load. Tissue conductivity and the dissipation factor (tan δ) of the dielectric substrate layer are also relevant factors. For example, if the composite relative permittivity is 20 and the dissipation factor has a low value of 0.001, then the loading factor will be approximately 20, i.e. √[20²+(0.001×20)²]=20.00001. The dimensions of each conducting patch may therefore be calculated taking these factors into account in order to generate a substantially uniform electromagnetic field at the treating surface.

The plurality of independently controllable power sources may permit the emitted electromagnetic field to be adaptable across the treatment surface. In other words, the radiation from the radiating elements may be adjustable. The field emitted by the device is therefore controllable e.g. to achieve beam steering and/or site specific focussing of the radiation. This is particularly useful for devices that cover a large area of tissue, since the impedance of tissue may vary over the treatment area due to the changes in biological tissue structure over the area that the applicator is in contact with.

Preferably, each power source includes a power amplifier and a monitoring unit arranged to detect the power delivered by the amplifier, such that the power supplied by the power amplifier is controlled on the basis of the power delivered into the biological tissue detected by the monitoring unit. The monitoring unit may also be arranged to detect the power reflected back to the power amplifier, so that the power supplied to the power amplifier is further controlled on the basis of the reflected power detected by the monitoring unit (i.e. power delivered into tissue=[demanded power−reflected power]). The monitoring unit preferably comprises forward and reverse directional couplers. These may be provided in a single device (a dual directional coupler) or as two single directional couplers. These units may take the form of microstrip couplers or waveguide couplers. This arrangement provides the ability to compensate for varying impedances over the area of tissue to be treated, e.g. due to moisture, tissue structure, etc., to control finely the level of energy radiated into the tissue and to focus the emitted field as a further means of control.

Preferably, the feed structure includes a primary stable microwave frequency energy source and a network of transmission lines for carrying energy from the primary energy source to the plurality of power sources and on to the radiating elements.

The network transmission lines may include a plurality of power splitters arranged to divide an output from the primary energy source into a plurality of inputs, each input being for a respective power source. The plurality of power splitters may include one or more buffer amplifiers arranged to compensate for power loss during the division of the primary energy source output.

To control the power supplied to its power amplifier on the basis of information detected by the monitoring unit, each power source preferably includes an dynamic impedance matching unit (i.e. impedance tuner) arranged to match the impedance of each radiating element to the skin tissue to be treated. In the present invention, impedance matching is preferably achieved electrically (as opposed to mechanically). Impedance matching may be achieved by phase adjustment (e.g. a PIN diode or varactor diode phase shifter. In the latter arrangement, the capacitance of the device is varied by applying a voltage to the device. Any matching filter (which can adjust the phase and magnitude of the signal supplied to the power amplifier) may be used to match the impedance of the system to that of the tissue (skin). These devices can be used e.g. if each radiating element is provided with its own power amplifier, so the power delivered through the network of transmission lines is limited to a maximum value e.g. of about 4 W. Small impedance matching devices, for example PIN diodes, cannot normally operate at the substantially higher power levels used with other types of treatment apparatus, where, for example, a single power source may deliver up to 120 W.

Since high frequencies are used in the present invention, physically small PIN phase shifters and microstrip directional couplers may be used as the dynamic impedance matching device and monitoring unit respectively. Such components can have a footprint (or surface area) of less than 5 mm², and in some cases less than 1 mm². By using small components, the device may comprise an integrated structure whereby the monitoring unit and dynamic impedance matching unit are located physically close to the power amplifier to minimise or at least reduce feed line losses. For example, the device may have a stacked layer structure. The layered structure proposed herein may involve vertically stacking layers with different functions on top of one another. The layered structure may reduce insertion loss or feed line loss between the power source(s) and the plurality of radiating elements, and may also enable the overall size of the device to be reduced. For example, the microwave sub-system may be contained within a block that has the same surface area as the applicator and the DC power supply and other associated low frequency instrumentation may be contained inside a separate unit that is located remotely, e.g. on a surface close to the patient.

It is preferable for all of the microwave components used for the power source to be integrated into a single layer. The stacked layer structure may include a first layer comprising the radiating elements disposed onto the dielectric substrate, a second layer comprising the monitoring and impedance adjusting devices for each radiating element (or groups of elements, for example, 2 or 4), a third layer comprising the power amplifiers for each radiating element (or groups of elements, for example, 2 or 4), and a fourth layer comprising the plurality of power splitters (these may be fabricated in the form of a network of transmission lines). Further layers comprising additional elements of e.g. a detector or receiver and a controller (discussed below) may also be provided. The compact nature of this structure may enable the device to be provided in a portable unit and the system may lend itself well for use in outpatient or home treatment.

The transmission lines may be shielded from the treating surface e.g. by being sandwiched in the dielectric layer located between the conducting ground plane and the conducting patches (stripline structure), or by being located on the opposite side of the conducting ground plane to the conducting patches (coplanar structure). The stacked layer structure is one way of achieving this shielding. Preferably, a coaxial connection connects each radiating element and the grounded conductive layer to a transmission line. For example, a wire or pin can be inserted through the dielectric substrate layer so that an electrical connection is made to the underside of a conducting patch. Static matching may be performed to cancel out a fixed reactance presented by the pin (the pin may exhibit inductive reactance). Thus, a stub that provides an equal value of capacitive reactance may be provided to give a conjugate impedance match.

The feed structure may be arranged so that at least one transmission line is arranged to deliver microwave energy from one or more of the power sources to a plurality of conducting patches connected in series. The plurality of radiating elements may be formed from a plurality of series-fed conducting patches. Each series may be formed by interconnecting all of its conducting patches, or radiating elements, with high-impedance transmission lines and feeding in power at one end.

Alternatively or additionally, the feed structure may be arranged so that at least one transmission line is arranged to deliver microwave energy from one or more of the power sources to a plurality of conducting patches connected in parallel.

Series arrays are preferred because the feed arrangement is more compact than the parallel (corporate feed) arrays, which means that the line losses (or insertion losses) are typically lower. The series (e.g. linear) arrays may operate in either a resonant or non-resonant mode.

Preferably, the feed structure is arranged to cause electromagnetic fields emitted by adjacent conducting patches to be orthogonal to one another. Thus, adjacent patches preferably radiate along edges that are orthogonal to each other. This facilitates uniform tissue effect over the whole treating surface area.

Preferably, the treating surface, radiating elements and feed structure are formed on a flexible sheet of dielectric material that is metallised on one or both sides and is conformable to the region of skin to be treated. This arrangement is particularly suitable for treating wounds where the treatment surface may be uneven or where it may be necessary to wrap the antenna around a region of the body, for example, a leg or an arm.

Preferably, the device includes a cover portion e.g. of dielectric material for locating between the treating surface and the region of skin to be treated. The cover portion may be a thin layer, i.e. a superstrate, mountable on a tissue facing surface of the patch antenna array. The cover portion may be arranged to enhance the uniformity of the field produced by the antennas by dispersing the fields produced by each of the radiating elements. The cover may also act as an insulation barrier between the radiating antennas and the surface of the skin, i.e. this may prevent any risk associated with the radiating elements (patches) causing burning to the surface of the skin by conductive heating caused by lossy structures (dielectric material, feed lines, and radiating patches contained within the antenna structure). Where a dynamic impedance matching unit is used, the radiation from each radiating element may be steered or shifted in phase further to improve field uniformity.

The cover portion may be formed from a block of one or more dielectric materials having different relative permittivities that are selected to slow down the electromagnetic waves. Alternatively, the cover portion may include upstanding dielectric posts arranged to ensure the presence of an air gap between the treating surface and the tissue to be treated. The air gap may be used to focus the electromagnetic field. The block or air gap preferably has a thickness of less than 0.1 to greater than 2 cm. Preferably, the block is made from a material that is low loss (i.e. low tan δ value, for example, 0.0001) at the frequency of interest. This is important for two reasons. Firstly it prevents a high portion of the microwave energy from being absorbed into the dielectric block. Secondly it prevents the block from heating up and causing burns on the surface of the skin due to the microwave energy being dispersed in the material causing it to get physically hot. The block may comprise or include a superstrate layer adapted to contact the tissue to be treated (again, it is preferable for the superstrate material to exhibit a low value of tan δ). Preferably, the superstrate is made from biocompatible material. The superstrate may be a conformal coating of biocompatible material e.g. Parylene C formed on the block. The coating is preferably of a thickness that makes it transparent to microwaves, e.g. 10 μm. Parylene C is particularly useful because it is relatively easy to apply as a coating. Preferably the dielectric block is made from a material with a high thermal conductivity, i.e. a ceramic material.

Using a cover portion that provides an air spacing or a low loss dielectric block between the radiating elements and the skin tissue may increase the Q value of the device because there is no damping caused by the tissue itself. In other words, separating the radiating patches from the skin tissue may mean that the reduction of the radiation's wavelength caused by the high relative permittivity of the skin tissue does not need to be taken account when determining the optimal for the size of the radiating elements, i.e. in the calculation of the half wavelength patch. This may also be advantageous in terms of matching the antenna to the varying properties of the skin due to a range of people and a range of locations over the body that are to be treated. Moreover, separating the radiating patches from the skin tissue can minimise unwanted heating of the tissue and reduce the risk of burning. This heating can be caused by the microwave transistors having a low microwave to DC power efficiency (i.e. 10 to 20%). Another way to reduce heating is to increase this efficiency by biasing the transistors to operate in a class other than the standard class A used, for example, in telecommunications where linearity is an important factor. For medical applications, pertinent factors may include the generation of appropriate power levels, the ability to generate power at a high enough microwave frequency to be useful, and optimisation of the efficiency of the device(s) that produce the power at the desired frequency. For example, the ratio of the output microwave power divided by the input DC power is preferably greater than 20% and more preferably greater than 50%, i.e.

$$( ( \frac{microwave output power}{DC input power} ) \times 100 ) > 50 \% .$$

For example, to achieve this, class A-B, class B, class D, class F or class S may be used. However, even if the transistors are operated in the non-optimal class A, so long as the radiating elements are not in contact with the skin, the heat generated by the transistors can be removed using known methods (e.g. Peltier coolers, fans, cooling pipes or water cooling). The device may operate in a pulsed mode where the duty cycle is low, for example, less than 10%, in order to reduce the average power dissipation, for example, operation using 10 W power levels with a 10% duty cycle implies that the average power over one cycle is 1 W.

Preferably, the cover portion is separable from the treating surface, whereby it may be used as a disposable element, which is usually necessary for clinical usage.

The combination of a suitably configured patch antenna arrays and impedance matched feed lines, together with new SHF or EHF semiconductor energy sources described above may therefore produce instantaneous and uniform tissue effects with depths of penetration and surface areas suitable for use in the treatment of a range of dermatological conditions. As demonstrated below, the device of the present invention permits treatment at a variety of penetration depths, which enables effective treatment of skin lesions at various stages of growth. Moreover, a variety of penetration depths made possible with SHF and EHF radiation also enables controlled coagulation of surface tissue for applications relating to skin removal (skin grafts or wound/tissue damage). Potential advantages of the new device include the reduction of pain (due to application of energy in short bursts, for example, 10 ms to 100 ms), alleviation of the need for bandaging, improvements in healing time, and prevention of bacteria from entering large areas of tissue where skin has been removed. It may be possible to use pulses that are of such duration that the brain does not receive any stimuli from the nerve endings, but, on the other hand, the tissue is able to respond in terms of causing a change in its biological state, i.e. does cause cell necrosis of the desired tissue structure being treated. Furthermore, this invention may enable treatment time to be reduced e.g. compared with conventional photocoagulation devices. Indeed, treatment may be given or delivered in a single dose.

Another advantage of the present invention occurs because of the linear relationship that exists between the number of radiating elements (conducting patches or other antenna structures) and the power delivered from the power sources when the radiating elements are fed correctly. This enables the treating surface to cover and treat uniformly relatively large areas of skin. For example, uniform tissue effect over a range of surface areas from less than 0.5 cm² to over 10 cm² may be possible, e.g. to enable various sizes of open wounds and exposed tissue following skin grafts to be sealed by controlled ablation, or to treat large areas of melanoma.

Preferably, the power amplifiers in the power sources are solid state semiconductor MMICs. The power amplifiers are preferably arranged to produce controlled energy in the super and extreme high frequency region of the electromagnetic spectrum. For example, the power amplifiers may operate at 14.5 GHz, 24 GHz, 31 GHz, 45 GHz, 60 GHz, 77 GHz or 94 GHz. Treatment systems operating at 31 GHz, 45 GHz, 60 GHz, 77 GHz and 94 GHz devices are made possible through recent advances in communication technology. Power generation at these frequencies may be realised using high electron mobility transistors (HEMTs), in particular indium phosphide based InAlAs/InGaAs HEMT structures. It may be possible to generate up to 4 W using a single PHEMT device that will operate up to 45 GHz. This power may be split to feed several patches or radiating elements, for example eight radiating elements may be excited e.g. using one 4 W device. Metamorphic HEMT (MHEMT) technology is another suitable candidate. These devices can generate power at frequencies at and in excess of 77 GHz.

As mentioned above, the device may include dielectric posts, or lengths of material attached around the edges of the treating surface to create an air gap between the treating surface and the region of skin tissue to be treated. The provision of an air gap during treatment may enable superficial tissue effects to be achieved, for example, skin resurfacing and/or skin rejuvenation. The present invention may also be usable for collagen shrinkage, hair removal or the treatment of alopecia areata due to the range of possible penetration depths. The air gap may also be used to focus or steer the emitted electromagnetic field, as described above.

In a second aspect, the present invention may provide apparatus for treating skin tissue with microwave radiation, the apparatus including: a source of microwave radiation having a stable output frequency or a range of selectable stable output frequencies; a treatment device as described above connected to the source of microwave radiation; and a controller arranged to control the amount of energy delivered via the microwave radiation to the tissue to be treated. Other devices used in the apparatus may include a microprocessor unit (e.g. including digital signal processor (DSP)) for control and monitoring, a user interface comprising a display and an input device (e.g. keyboard and/or mouse or touch screen display), a DC power supply unit, and a suitable housing. The microprocessor unit is preferably arranged to receive the detected information from the monitoring units associated with each radiating element(s) and to control the respective dynamic impedance matching units accordingly.

In a third aspect, there may be provided a method of treating skin tissue with microwave radiation, the method including: covering a region of skin to be treated with a treating surface that has a plurality of radiating elements thereon; connecting a source of microwave radiation having a stable output frequency or a range of selectable stable output frequencies in the EHF or SHF range to the radiating elements via a plurality of independently controllable power sources, whereby the radiating elements emit a microwave electromagnetic field which penetrates the region of skin to be treated to a predetermined depth; and controlling the power delivered by the power sources to the radiating elements to permit uniform energy delivery over the region of skin to be treated.

When used at frequencies towards the higher end of the spectrum disclosed herein, the invention may be used to treat skin viruses or other types of virus found in skin tissue. The invention may enable the DNA structure of the virus to be changed e.g. to deactivate the virus. This method of treatment may have advantage over antibiotics where the body becomes resistant and the particular antibiotic has no effect. The body will not become immune to the treatment system described herein.

The invention may be also used for the treatment of benign skin tumours e.g. actinic keratosis, skin tag, cutaneous horn, seborrhoeic keratosis, or general warts. A particularly relevant clinical application that is of interest in relation to the invention may be the treatment of atopic and seborrhoeic dermatitis or acne, where over-activity of the sebaceous or sweat glands cause excessive sweating, which can lead to bacteria or fungus forming on the surface of the skin. The fungus produced is known as pityrosporum, which is a common bacterium that forms on the skin and manifests in regions where people sweat, for example, the head, under the breast, the forehead, and the armpits. Since people with seborrhoeic dermatitis produce more sweat than normal this leads to more pityrosporum fungus being produced. A microwave or millimetre wave power source activated to deliver power via radiating elements (for example a 10 mm² patch, or an array of patch antennas) at the skin surface to deliver a controlled dose of energy into the sebaceous gland may inhibit the excessive activity.

The new skin system proposed here may be effective for treating all structures of the skin, and, if this is the case, it could be useful not only for the skin cells but also for the blood vessels, the nervous system and even for the immune system of the skin. The system may, therefore, be effective for treating the following conditions that relate to the skin: pyoderma gangrenosum, vitiligo, prurigo, localized morphea, hypertrophic scar and keloid etc.

The treatment system described here may also be used for relief of chronic pain, i.e. postherpetic neuralgia (PHN).

Another potentially relevant clinical application is the treatment of alopecia areata. Alopecia areata is an autoimmune disease where the body's immune system mistakenly attacks hair follicles, which are the part of skin tissue from which hairs grow. If this condition arises, the hair normally falls out in small round patches. This condition may be treatable through the stimulation of hair follicles using high frequency microwave or mm-wave energy. According to the invention, this energy may be supplied via an array of patch antennas that can be stuck onto the scalp. The range of sizes of the patches or arrays may be developed to accommodate the amount of hair loss caused by alopecia in a particular patient, for example, the size may range from 1 cm² to 100 cm². This treatment of alopecia areata may require a small depth of penetration e.g. around 0.1 mm, thus this invention may lend itself particularly well to this clinical application when frequencies in excess of 100 GHz, for example, 300 GHz or more are used. The material used to carry or house the antennas may be a flexible or conformable material that makes good contact with the scalp. Each antenna in the array may be fed energy from a separate amplifier or power splitters may be used to deliver the power into each antenna to cause it to radiate the appropriate amount of energy into the scalp.

### BRIEF DESCRIPTION OF THE DRAWINGS

Other features of the invention are explained in the detailed description of examples of the invention made below with reference to the accompanying drawings, in which:

FIGS. 1(a), 1(b) and 1(c) show a treatment system that is an embodiment of the invention adapted for treating skin lesions;

FIGS. 2(a), 2(b) and 2(c) show a treatment system that is another embodiment of the invention adapted for treating open wounds;

FIG. 3 is a cross-sectional view through a skin treatment device which is a further embodiment of the invention;

FIG. 4 is a block diagram illustrating an entire skin treatment apparatus which is a further embodiment of the invention;

FIG. 5 is a schematic representation of the stacked layer structure that can be implemented in an embodiment of the invention;

FIG. 6 illustrates the feed structure of apparatus shown in FIG. 4;

FIG. 7 illustrates a single monitoring unit from the apparatus shown in FIG. 4;

FIG. 8 shows a schematic view of a skin treatment device that is another embodiment of the invention;

FIGS. 9(a), 9(b) and 9(c) show a top view, bottom view and side view of a skin treatment device that is yet another embodiment of the invention;

FIG. 10 shows an example of a feed structure for providing power to radiating patches in a device according to the invention;

FIG. 11 shows an example of a feed structure which provides power from amplifiers in one layer in a device to radiating patches in another layer of that device;

FIG. 12 is a cross-sectional view of the arrangement shown in FIG. 11;

FIG. 13 is a schematic view of a first feed arrangement that can be applied to the present invention;

FIG. 14 is a schematic view of a second feed arrangement that can be applied to the present invention;

FIG. 15 is a schematic view of a third feed arrangement that can be applied to the present invention;

FIG. 16 is a schematic view of a fourth feed arrangement that can be applied to the present invention;

FIG. 17 is a plan view of a practical embodiment of the feed structure shown in FIG. 16;

FIG. 18 is a plan view of an array of patch antennas for use with 14.5 GHz radiation;

FIG. 19 is a plan view of an array of patch antennas for use with 31 GHz radiation;

FIG. 20 shows a feed structure with buffer amplifiers that can be used in an embodiment of the invention;

FIG. 21(a) shows the cross-section of a conventional coplanar waveguide structure feeding a single suspended patch antenna;

FIG. 21(b) shows the cross-section of a grounded co-planar waveguide structure feeding a single suspended patch antenna;

FIG. 22(a) shows an alternative view of a single patch antenna suspended in air using a feeding post connected between the radiating antenna patch and the coplanar waveguide structure;

FIG. 22(b) shows an array of suspended patch antennas fed using coplanar waveguide lines where the ground plane of the coplanar waveguide also provides the ground plane for the radiating patch antenna; and

FIG. 23 shows a specific embodiment of the antenna array and microwave sub-assembly that uses an array of sixteen radiating suspended patch antennas fed using a co-planar waveguide structure together with an arrangement of microstrip lines.

### DETAILED DESCRIPTION

### Further Options and Preferences

The general principle of the present invention is the production of electromagnetic radiation with a substantially uniform field from an array of radiating elements. In some of the embodiments described below, patch antennas are used as the radiating elements. Arrays of slotted lines or coplanar waveguide fed suspended patches may also be used. Micromachining technology can be used to fabricate such radiating elements and their feed line structures. A further embodiment provides a radiating structure comprising a bottom layer with a plurality of slots in a ground plane and an arrangement of microstrip lines fabricated onto a dielectric layer such that the radiating microstrip lines are over the slots. The microstrip lines and slots are sized such that energy is radiated from the slots. The operating environment for the patch antenna arrays introduced here is very different from the usual ‘free space’ conditions where such antenna structures are normally operated. For example, arrays of patch antennas are normally employed in ship radar, ground radar, and various other types of communications equipment, hence biological tissue presents a somewhat unconventional environment for the arrays of patch antennas to operate, since the structures in the present invention will normally operate in the near field, i.e. the operation may be considered to be capacitive coupling between the antenna and the tissue, where displacement currents are involved.

Operating in a biological environment presents particular challenges. The high dielectric constants associated with the skin tissue will cause resonant structures to be reduced in size relative to free space. For example, for treatment of wet skin, a patch, or half-wave dipole antenna element, will be about 1.16 mm² at 31 GHz, whereas in air it is 4.8 mm². Thus, the geometry of the resonant patch antenna structures may need to be adjusted in order to preserve resonant operation so maximum energy is delivered (i.e. energy is delivered with optimum efficiency).

To ensure uniform radiation over a large area, measured in terms of wavelengths, a large number of patches are used. Due to the high local conductivity of the skin tissue, the usual resonant behaviour of patch array antennas will be lost. This limits the control over impedance and the ability to match to the feed distribution networks. For example, the input impedance of a quarter-wave monopole may fall from 35Ω to 5Ω. Thus, additional matching may be required to match the feed structure to the radiating patches. A dynamic impedance matching unit may be required to achieve this. A possible arrangement is described below.

Table 1 provides a list of the relevant electrical and dielectric properties associated with dry and wet skin. These properties are taken into account when designing the patch antenna arrays to ensure that the patches efficiently radiate energy into skin tissue, and produce a uniform effect on the tissue over the whole surface area of the device.

TABLE 1
Tissue Parameters for Dry and Wet Skin over a range
of microwave frequencies from 5 GHz to 100 GHz

| Frequency | Dry skin | | | Wet skin | | |
|---|---|---|---|---|---|---|
| GHz | σ (S/m) | εᵣ | d (mm) | σ (S/m) | εᵣ | d (mm) |
| 5 | 3.06 | 35.77 | 10.49 | 3.57 | 39.61 | 9.49 |
| 10 | 8.01 | 31.29 | 3.80 | 8.95 | 33.53 | 3.53 |
| 14.5 | 13.27 | 26.88 | 2.16 | 14.08 | 28.62 | 2.10 |
| 20 | 19.22 | 21.96 | 1.38 | 19.71 | 23.77 | 1.39 |
| 30 | 27.10 | 15.51 | 0.85 | 27.52 | 17.74 | 0.88 |
| 31 | 27.69 | 15.030 | 0.82 | 28.151 | 17.294 | 0.85 |
| 40 | 31.80 | 11.69 | 0.65 | 32.87 | 14.09 | 0.67 |
| 45 | 33.94 | 10.40 | 0.59 | 34.94 | 12.81 | 0.605 |
| 50 | 34.62 | 9.40 | 0.54 | 36.69 | 11.77 | 0.56 |
| 60 | 36.40 | 7.98 | 0.48 | 39.52 | 10.22 | 0.49 |
| 70 | 37.58 | 7.04 | 0.43 | 41.71 | 9.12 | 0.43 |
| 80 | 38.40 | 6.40 | 0.40 | 43.46 | 8.32 | 0.40 |
| 90 | 38.99 | 5.94 | 0.38 | 44.90 | 7.72 | 0.37 |
| 100 | 39.43 | 5.60 | 0.36 | 46.12 | 7.25 | 0.35 |

The symbols given in the table above: ∈ᵣ, σ and d represent relative permittivity (dimensionless), conductivity (Siemens per metre) and depth of penetration (millimetres) respectively. Electromagnetic field modelling packages, for example, Computer Simulation Tools (CST) Microwave Studio®, were used to model the antenna array structures considered in this work.

The frequencies that are investigated in the embodiments described below are: 14.5 GHz, 31 GHz and 45 GHz, where the depths of penetration in dry and wet skin are 2.16 mm and 2.10 mm respectively at 14.5 GHz, 0.82 mm and 0.85 mm respectively at 31 GHz, and 0.59 mm and 0.61 mm respectively at 45 GHz. Similar techniques may be applied to devices operating at higher frequencies (e.g. 60 GHz, 77 GHz or 94 GHz). These frequencies are the preferred operating frequencies for the treatment applicators considered in this invention due to the fact that the depths of penetration produced are of interest for treatment of a number of conditions related to the skin; these frequencies lie within the regions of the microwave spectrum known as the ‘super high frequency’ region (SHF) and the ‘extremely high frequency’ region (EHF). Due to the fact that the associated wavelengths are small compared to lower microwave frequencies, it is possible to produce a large array of single-wavelength or half-wavelength radiating patches in a relatively small surface area to help ensure uniform tissue effects are obtainable. Devices operating at higher frequencies can be used where smaller penetration depths are required.

The combination of small radiation penetration depth and the ability to manufacture radiating patches with small surface areas makes possible the practical use of energy sources operating at these high microwave frequencies for dermatological applications.

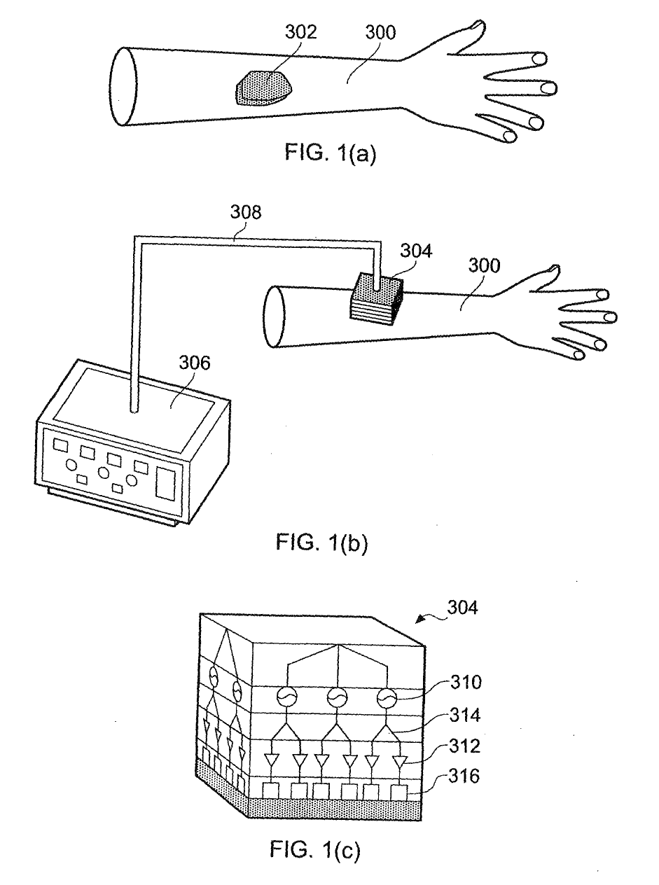

FIGS. 1(a), (b) and (c) shows an illustration of the complete treatment system that may be used for treating a cancerous lesion on the arm of a patient. FIG. 1(a) shows an arm 300 with a lesion 302. FIG. 1(b) shows a radiating antenna array 304 treating the lesion 302. The overall treatment system comprises two sub-systems 304, 306 that are connected together using a cable assembly 308 which contains transmission lines for the DC power supplies and the transmission lines for control signals. The operating frequency for the control signals is very low compared to the microwave frequency spectrum, for example, between 1 Hz and 100 KHz, thus the insertion loss along the cables is negligible and a range of standard cables, for example seven strands of 0.2 mm (7/0.2 mm) diameter tinned copper wire may be used. The first sub-system 306 contains a DC power supply, a control unit (e.g. a microprocessor and/or a digital signal processor) and an appropriate user interface (e.g. a keyboard/mouse with a monitor, a LED/LCD display with a keypad or a touch screen display or similar). The second sub-system is the microwave sub-assembly 304, shown in detail in FIG. 1(c), which contains a microwave source oscillator(s) 310, microwave power amplifiers 312, a power splitting and feed network 314, and a radiating antenna array 316 (all described in more detail below). This unit also includes directional couplers (not shown), for example microstrip couplers, detectors, and a means of dynamic tuning or beam steering. The directional couplers are used to enable levels of forward going, or reflected, power to be monitored, and the signals from the coupled ports of said couplers may be used to control PIN diode phase shifters or variable capacitance varactor diodes (also not shown) to enable the antenna array to be impedance matched to the surface impedance of the skin.

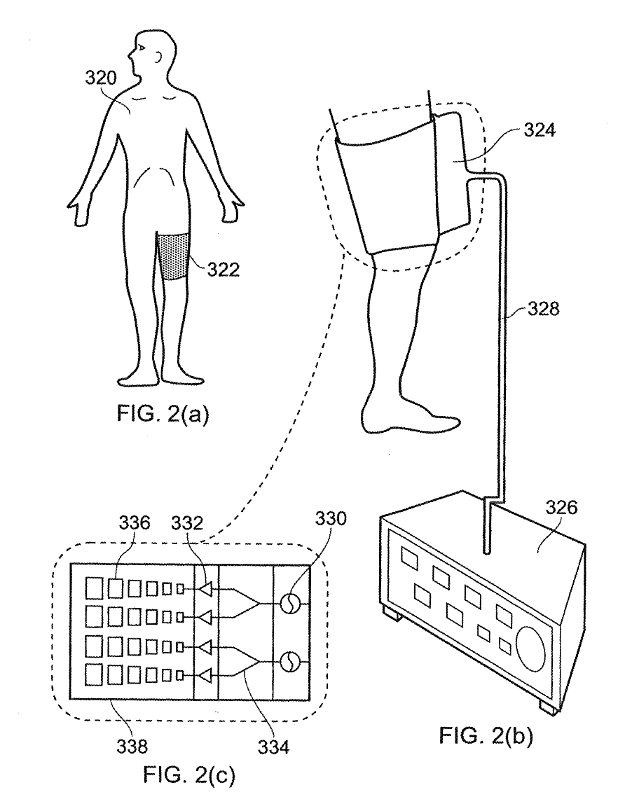

FIGS. 2(a), (b) and (c) show an illustration of a system used to treat a large wound to the leg of a patient. FIG. 2(a) shows a patient 320 with a large open wound 322 on his or her leg. This wound may be caused, for example, by a skin disease, a car accident, or through being involved in a battle or a war. FIG. 2(b) shows the complete treatment system, which includes two sub-systems 324, 326 that are connected together using a cable assembly 328 containing the transmission lines carrying DC power supplies and transmission lines carrying control signals. The first sub-system 326 has a DC power supply, a control unit (e.g. a microprocessor and/or a digital signal processor) and an appropriate user interface (e.g. a keyboard/mouse with a monitor, a LED/LCD display with a keypad or a touch screen display). The second sub-system is the microwave sub-assembly 324, which is shown in more detail in FIG. 2(c). The microwave sub-assembly 324 contains microwave source oscillator(s) 330, microwave power amplifiers 332, a power splitting network 334, and radiating antennas 336. In this embodiment, the radiating antennas 336 are fabricated onto a flexible substrate 338 to enable it to be wrapped around the leg (or other region of the body with a similar structure). The microwave power amplifiers 332, the source oscillators 330, and the other microwave electronic components associated with the microwave sub-assembly 324 are desirably connected directly to the inputs of the flexible antenna array structure to minimise insertion loss.

In this embodiment, a plurality of travelling wave antenna structures are used to form the flexible antenna array.

In practice, two antenna arrays of the type shown in FIG. 2(c) may be used together to enable the system to produce uniform tissue effects necessary for fast wound healing around the complete circumference of the leg. It may be desirable to use more than two arrays where larger surface areas are to be treated.

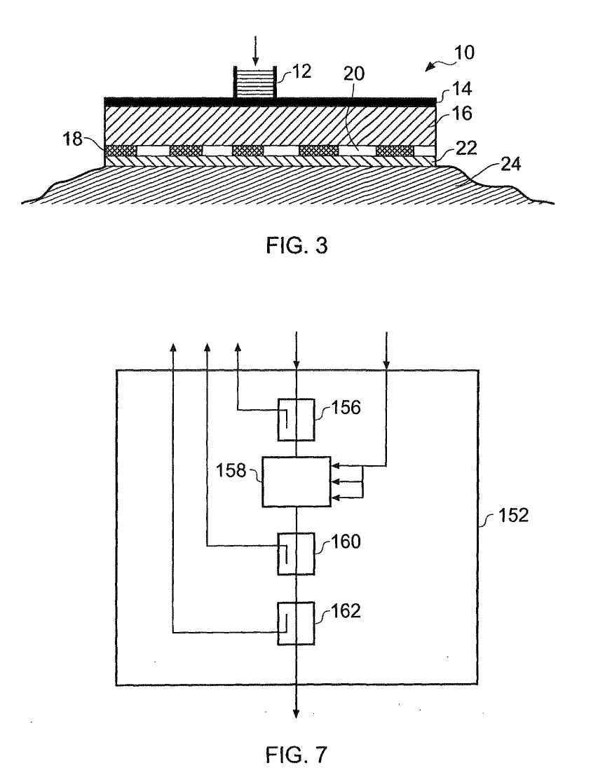

FIG. 3 shows a skin treatment device 10 which is an embodiment of the present invention applied to a skin surface 24. The device 10 has a microwave feed connector 12 through which energy e.g. AC power having a predetermined stable frequency is provided to the device from an energy source (not shown). The feed connector may be any suitable type, e.g. a coaxial connection such as SMA, SMB, SMC, MCX or SMP. A grounded conductive layer 14 (e.g. of copper, silver or the like) is mounted on the dielectric substrate 16 to provided a return current path for current supplied to a plurality of conducting patches 18 via a feed structure (discussed below). Each patch 18 has a rectangular shape selected so that it acts as a radiating antenna for the provided microwave energy. The shape of the radiating elements is not necessarily rectangular, i.e. they may be square, triangular or cylindrical. The shape may be optimised using an electromagnetic field simulation. The plurality of patches 18 are arranged in a regular array, separated by air gaps 20 on the surface of substrate 16 so that together they emit outwardly a substantially uniform electromagnetic field. The array of patches 18 are covered by a dielectric superstrate 22, preferably formed from a biocompatible material, e.g. Parylene C, Teflon® or the like.

Typically, the superstrate 22 contacts the skin 24 during treatment. However, if more superficial treatment is required (e.g. for tissue resurfacing), an air gap may be introduced between the superstrate 22 and skin 24. If the distance between said air gap and said tissue is such that signal attenuation is less than 1 dB for example, then it is possible to couple a significant portion of the source energy into the surface of the tissue without having to place the surface of the applicator directly in contact with the surface of the tissue. The advantages of this method of treatment are: there should be no possibility that the surface of the tissue can be damaged in terms of burning or tissue carbonisation due to a hot applicator, and the energy distribution may be altered by adjustment of the stand-off distance, e.g. by having an adjustable threaded engagement between one or more dielectric posts protruding from the device. This method can be used to affect tissue beneath the surface of the skin, whilst leaving the skin surface unaffected. Particular applications may include collagen shrinkage and the destruction of clusters of hair follicles.

Alternatively, a low loss dielectric block may be used between the radiating patches and the surface of the skin. The energy adjustment may also be made by adjusting a PIN diode attenuator to control the power level, or by modulating a PIN diode switch to change the pulse width or the duty cycle of the energy delivered. Alternatively, PIN diode phase adjusters may be used to control the phase of the radiating patches with respect to one another. A combination of adjustment of power level delivered to individual patches (or radiating elements) and an adjustment in phase will enable uniform energy to be delivered into the surface of the skin over large surface areas when changes in the structure of the tissue—both on the surface and below the surface—may require different amounts of energy or different matching conditions. Thus, the present invention may provide individually controllable radiating elements which can adapt to variability in tissue structure over a treatment area.

The superstrate 22 is removable, and forms the disposable part of the apparatus.

The dielectric substrate 16 may be of any suitable material, i.e. dielectric material preferably with a low tan δ and a relative permittivity that helps to impedance match the device to the surface of the skin tissue being treated. Examples of suitable materials are: PTFE, nylon, sapphire, and alumina coated with Parylene C (where the thickness of the coating is preferably less than 10 μm). Advantages of using alumina include having a relative permittivity of around 10, which is comparable to that of the skin structure, and having good thermal conductivity. In certain instances it may be desirable to use a material with a poor thermal conductivity in order to prevent any heat generated by conduction from being transferred to the surface of the tissue, which could result in burning of the surface of the tissue, i.e. the heat will be stored in the material rather than being conducted into the skin.

The relative permittivity of PTFE or nylon tends to be relatively low, for example, between 2 and 4, thus a matching transformer may be required between the dielectric substrate layer and the patch antenna layer. In the instance where a low permittivity dielectric is used, it is preferable to sandwich an additional dielectric layer between the dielectric substrate layer and the patch antenna layer to perform the necessary impedance matching and to prevent a portion of the power being reflected at the tissue/dielectric interface.

If it is required to keep the surface of the skin cool whilst treating diseased skin tissue, the patch antenna array could be mounted on a Peltier cooler device. This may be of particular interest for collagen shrinkage applications. A ceramic substrate with good thermal conductivity may also help to remove heat from the surface of the skin.

It may also be possible to spray the surface of the skin with a coolant or freezer spray to cool the surface of the tissue when the microwave energy is applied. In this arrangement, the microwave energy is absorbed inside a layer or layers of the skin to a depth that is related to the frequency of the microwave energy, and the surface of the skin is unchanged. It may be preferable to synchronise the delivery of the coolant with the application of the microwave pulses. For example, if the microwave pulse is of duration 100 ms, it may be desirable to activate the spray 50 ms prior to the pulse.

The structure illustrated in FIG. 1 is rigid and flat, but can be modified to produce a flexible array which conforms to irregular tissue structures. For example, Rogers Corporation and Sheldahl (now Multek Flexible Circuits) manufacture flexible laminate polymer circuit materials (e.g. Rogers Corporation produce a specific material known as R/flex 3600) which may be used in implementing the present invention.

Where conducting patches 18 are used, the device design is based on the theory of patch antenna arrays, where the size (length ‘L’ and width ‘W’) of each radiating patch is calculated as a function of the effective dielectric constant, which depends on the frequency of operation (e.g. 14.5 GHz) and the dielectric constant ∈ᵣ of the material used to fabricate the patch array, the dielectric constant of the skin tissue which the patch antenna is used to treat and the dielectric constant of the dielectric block or air gap (if used). The superstrate 22 will also affect the performance of the overall antenna structure and this has to be taken into account when designing and optimising the patch antenna array. If the thickness of the superstrate material is small, e.g. 5-10 μm, then the effect may be negligible and can be ignored. It is also possible to use a material that is relatively lossy, i.e. has a tan δ of greater than 0.001, if only a very thin layer is used.

The change in the effective dielectric constant due to a thick superstrate 22 may present a substantial change, and the amount of change is governed by the thickness and the relative permittivity of the superstrate 22.

Table 2 provides information based on ideal calculations performed to ascertain the number of patches per cm² for the dielectric loading associated with dry and wet skin with the applicators in contact with the surface of the skin. These figures assume that the radiating patches are in direct contact with the skin and that the substrate material on which the radiating patches are fabricated has no effect on the size of the patches. It also assumes that the component of permittivity due to the material loss is low compared to the relative permittivity. To obtain more accurate figures and/or take account of the factors ignored above, an electromagnetic field simulation can be carried out to enable optimisation of the size of the patch array or other antenna structures that are appropriate for use with the current invention to be performed.

TABLE 2
Idealised parameters associated with patch arrays
focussed into wet and dry skin tissue at frequencies
of 14.5 GHz, 31 GHz and 45 GHz.

| | Tissue Type | | | | | |
|---|---|---|---|---|---|---|
| | Wet Skin | | | Dry Skin | | |
| | Patch | | | Patch | | |
| | size | Patches | | size | Patches | |
| Frequency | L(W) | per 10 | Penetration | L(W) | per 10 | Penetration |
| GHz | (mm) | mm² | depth (mm) | (mm) | mm² | depth (mm) |
| 14.5 | 1.93 | 9 | 2.1 | 2.0 | 9 | 2.16 |
| 31.0 | 1.16 | 36 | 0.85 | 1.21 | 25 | 0.82 |
| 45 | 0.93 | 49 | 0.61 | 1.0 | 49 | 0.59 |

Solid state transistor devices that operate at the above frequencies are commercially obtainable from TriQuint Semiconductor, Toshiba Semiconductor, Hittite Microwave Components and Mitsubishi Semiconductor. Devices operating at 14.5 GHz are becoming well established, whereas devices operating at 31 GHz, 45 GHz, 60 GHz, 77 GHz and 94 GHz are now beginning to become available. TriQuint Semiconductor now manufacture 4 W devices that operate at 45 GHz and 31 GHz. With this power output, a single device may be used to feed a number of radiating elements. Recent developments in semiconductor technology, particularly in PHEMT devices provide power levels from 100 mW to 2 W to be generated at frequencies up to 100 GHz.

The figures given in table 2 have been rounded up or rounded down to enable complete half wavelength loaded patches to be accommodated in a square of surface area 10 mm². In practical implementations, the sizes may be slightly extended or reduced in order to optimise the number of patches that can be fabricated on the area of substrate material available, and the sizes can change in accordance with results obtained from electromagnetic field modelling. For example, if the dimension were to be increased to 10.62 mm (W) by 10.62 mm (L) then 16 complete half wavelength patches could be used in the array with an operating frequency of 14.5 GHz. These dimensions will change when simulations are performed since the interaction between the lossy biological tissue structures and the antenna structures will be taken into account. At the simplest level, there are three values of permittivity associated with the overall structure. These are:
the complex permittivity of the biological tissue (skin),
the complex permittivity of the superstrate layer, and
the complex permittivity of the substrate layer.
   It is possible to increase the number of patches in a uniform manner in order to increase the treatment area, for example, at 31 GHz, 144 patches could be used to fabricate a square treatment applicator with a surface area of 4 cm², therefore 576 patches would be required to fabricate a square treatment applicator with a surface area of 16 cm²

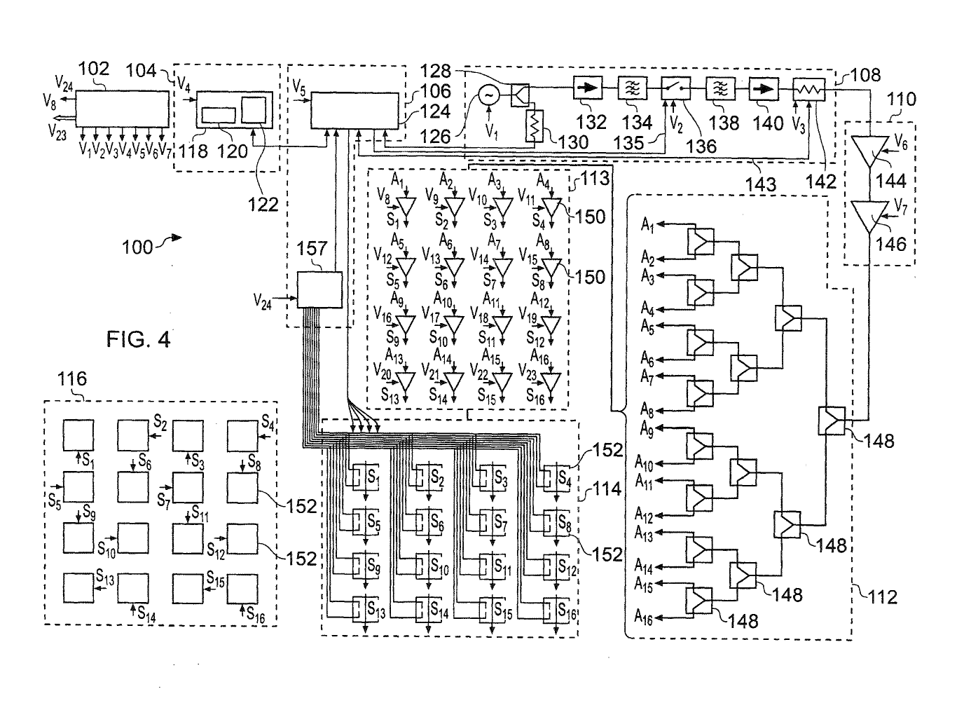

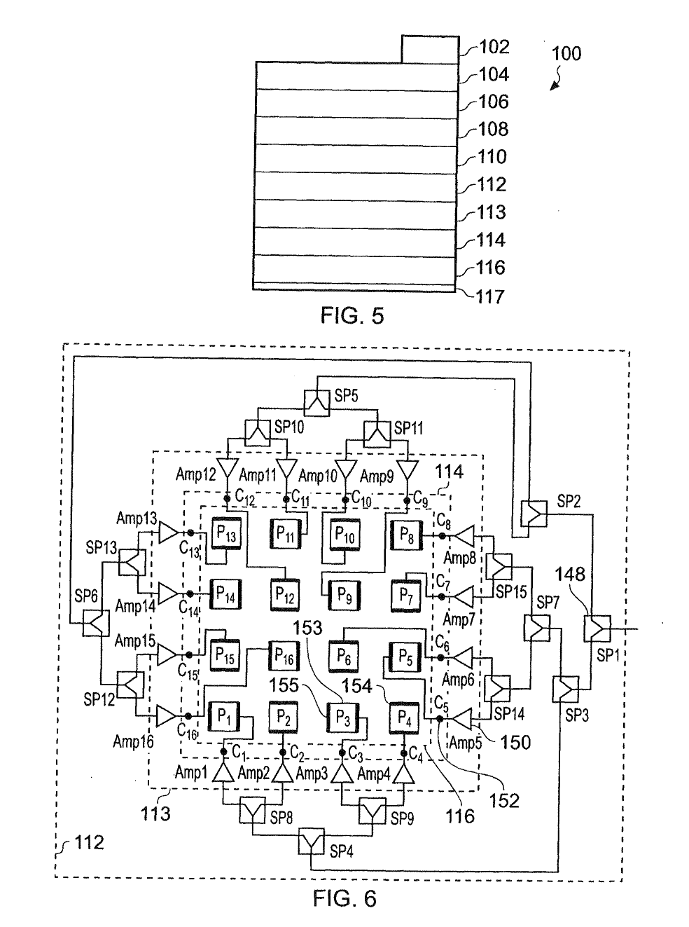

FIG. 4 shows a diagram of the components contained in a complete treatment apparatus 100 according to an embodiment of the invention. FIG. 5 shows a schematic representation of that apparatus wherein all of the apparatus components used for the microwave energy source, power feed structure and radiating antenna array are integrated onto a single substrate, thereby creating a compact overall design. Using vertical stacking techniques, the apparatus 100 is made up of a plurality of layers. A battery or AC/DC converter (i.e. a power supply) 102 is mounted on a first layer 104 which includes a user operable control and display device. The first layer 104 is mounted on a second layer 106, which includes a processor for the controlling the apparatus. This layer may also contain a second processor, known as a ‘watchdog’, which is used to monitor fault conditions and act as a means of protection in the instance that the first processor malfunctions. The second layer 106 is mounted on a third layer 108, which includes a microwave signal generating line-up. The third layer 108 is mounted on a fourth layer 110, which includes a microwave amplifier line-up (e.g. a plurality of MMIC or MHEMT devices) for boosting the generated microwave signal. The fourth layer 110 is mounted on a fifth layer 112 which includes a feed structure (e.g. of microstrip tracks) incorporating a network of power splitters arranged to divide the generated microwave signal and transmit energy to the radiating elements. The fifth layer 112 is mounted on a sixth layer 113, which includes an array of power amplifiers (e.g. MMIC devices) for boosting the divided signals before they are provided to the radiating elements of the antenna structure. The sixth layer 113 is mounted on a seventh layer 114, which includes an array of signal control devices arranged to monitor the power delivered to and reflected from each radiating elements and to adjust each signal e.g. to ensure impedance matching with the tissue to be treated. The seventh layer 114 is mounted on an eighth layer 116, which includes an array (e.g. regular pattern) of radiating elements (e.g. conducting patches, slot lines, or coplanar waveguide suspended patch antennas) that each receive a divided signal from the array of signal control devices. The eighth layer may have a grounded conductive coating on a surface opposite the radiating elements to provide a radiating arrangement similar to that shown in FIG. 4. A biocompatible removable (disposable) ninth layer 117 is provided on the eighth layer 116. The ninth layer 117 contacts the tissue to be treated during use (i.e. it is the superstrate layer described above).

Thus, the complete apparatus can be contained within a sandwich of layers. The main advantage of mounting the power devices directly onto the radiating patch is that transmission loss (or feed line loss or insertion loss) is minimised. This is of particular interest for high frequency (e.g. 24 GHz, 31 GHz, 45 GHz, 60 GHz, 77 GHz, 94 GHz and above) operation. It may be desirable to split the overall treatment system in two separate blocks as shown in FIGS. 1 and 2. The first block may contain the microwave sub-assembly, consisting of the superstrate layer, the antenna array, the feed structure, the power generating devices and the source oscillator(s). The second block may contain the DC power supply, the control electronics (microprocessor and/or DSP and/or watchdog) and the user interface.

The components in each layer are illustrated in FIG. 4. The microwave signal is generated by a stable frequency source 126, which provides a signal at a single frequency contained within the super high frequency (SHF) or extremely (EHF) region of the electromagnetic spectrum and, more specifically, at 14.5, 24, 31, 45, 66, 77, or 94 GHz (with a frequency variation limited to a few hundred kHz). The stable frequency source 126 shown here takes the form of a phase locked dielectric resonator oscillator (DRO), which contains a reference signal to which the frequency stability of microwave source 126 is derived; the source of said reference signal (not shown) may comprise a temperature stable crystal oscillator, operating at a frequency in the range of between 1 MHz and 100 MHz, but more preferably between 10 MHz and 50 MHz. Other frequency sources, such as a voltage controlled oscillator (VCO) or a Gunn diode oscillator may be used, but it is preferable to use a DRO in the present invention. Two reference oscillators can be used within microwave source 126 to enhance frequency stability of the system. It may be preferable to use a plurality of stable frequency sources to enable a plurality of microwave frequency sources to be used to excite a single patch antenna array. In this arrangement, a the stable frequency source may take the form of a frequency synthesiser.

The stable frequency source 126 is connected to the input port of a 3 dB, 0° power splitter 128. The purpose of splitter 128 is to divide the power produced by source 126 into two equal ratios without introducing a phase change.

The first output from splitter 128 is connected to the input of a first signal isolator 132, and the second output from splitter 128 is connected to the input of attenuation pad 130. The output of attenuator pad 130 is input into a microprocessor 124 where the signal is used to monitor the status of the frequency source 126. The purpose of attenuator pad 130 is to limit the signal level incident at the input to the microprocessor 124. Should the signal indicate that signal source 126 is functioning improperly, then microprocessor 124 will flag up that an error has occurred and the system will take appropriate action, i.e. an error message will be generated, and/or the system will be shut down.

The purpose of first signal isolator 132 is to prevent any mismatched signal present at the input of first modulation breakthrough blocking filter 134 causing frequency changes at source 126, due to, for example, load pulling, or another condition that may affect the signal generated by signal source 126. In practice, isolator 132 may not be required if the input port of filter 134 is well matched, but isolator 132 is included as a precautionary measure. The output of first modulation breakthrough filter 134 is connected to the input of a modulation switch 136, whose function is to modulate the signal produced by stable frequency source 126 to enable the system to operate in pulsed mode, whereby the duty cycle, pulse width, and (if wanted) pulse shape can be modified using the user control and display unit 118 and the microprocessor 124. The purpose of the first modulation breakthrough filter 134 is to prevent frequency components contained within the fast switching signals produced by the modulation switch 136 from getting back to the stable frequency source 126 and affecting its output signal.

An input control signal 135 to the modulation switch 136 comes from the microprocessor 124. This control signal 135 may be a transistor-transistor logic (TTL) level signal; other signal formats (e.g. emitter coupled logic (ECL)) are possible.

The output from the modulation switch 136 is connected to the input of a second modulation breakthrough blocking filter 138, whose function is to prevent frequency components contained within fast switching signals that may be produced by modulation switch 136 for certain treatment modalities from getting into the subsequent pre-amplifier 144 and power amplifier 146 and causing, for example, signal distortion, erroneous output power levels, or damage to these units through, for example, the manifestation of output power stage oscillation, or signal overdrive caused by one of the harmonics contained within the switching signal occurring at the same frequency as that of the signal generated by the frequency source 126 or a signal that is within the bandwidth of the amplifiers 144, 146, i.e. where said amplifiers provide gain.

A practical implementation of the breakthrough blocking filter may simply be a rectangular waveguide section, where frequencies lower than the cut-off frequency of the waveguide section will be blocked, hence the waveguide section acts as a high pass filter.

The output from second modulation breakthrough blocking filter 138 is connected to the input of a second signal isolator 140. The output from said second isolator 140 is connected to a variable signal attenuator 142, whose function is to enable the system power level to be controlled by changing the level of signal attenuation using input control signals 143 produced by microprocessor 124. Variable signal attenuator 142 may be an analogue or digital attenuator and may be reflective or absorptive type. This attenuator may be controlled by microprocessor 124 to produce a number pulse shapes or sequences. The function of the second signal isolator 140 is to provide isolation between the input port of the variable attenuator 142 and the output port of the second modulation breakthrough blocking filter 138. The second signal isolator 140 is inserted for good design practice and may be omitted from the apparatus without causing degradation or damage to the microwave sub-assembly.

The output from variable attenuator 142 is connected to the input of signal pre-amplifier 144, whose function is to amplify the signal to a level that is acceptable for driving the input to the subsequent power amplifier stage 146. The preamplifier 144 may provide a gain of between 10 dB and 40 dB necessary to drive the power amplifier stage 146. The preamplifier 144 may come in the form of a single miniature microwave integrated circuit (MMIC), a plurality of MMICs, a combination of MMIC(s) and discrete parts, or a plurality of discrete parts. MMIC devices are preferable to discrete parts since these devices normally produce more gain, hence a single MMIC may be used instead of a cascade of discrete parts; this is advantageous in terms of space (size) minimisation and heat dissipation. For example, TriQuint's semiconductor device TGA8658-EPU-SG can be used. The preferred device technology for use in the pre-amplifier is gallium-arsenide (GaAs) technology, although there are other emerging technologies that may provide viable alternatives, for example, gallium nitride (GaN) or high electron mobility transistors (HEMTs).

The output from pre-amplifier 144 feeds the input to power amplifier 146, whose function is to boost the signal to a level needed to supply the radiating antenna structure of the treatment device.

The output from power amplifier 146 is fed to a network of 3 dB power splitters 148. The power splitters 148 can be fabricated as a microstrip structure on their respective layer 112 of the apparatus. As shown in FIG. 6, the power splitting network comprises fifteen power splitters SP₁-SP₁₅ which divide the signal from the power amplifier into sixteen feeds A₁-A₁₆, each of which is connected to a respective amplifier 150 in the next layer 113. Thus, in this embodiment the amplifier network is fed from a single source.

Each of the sixteen amplifiers 150 is arranged so that its output drives a conductive radiating patch or antenna 154. The sixteen amplifiers 150 produce drive signals S₁-S₁₆ for this purpose. The amplifiers 150 each produce power at the 1 dB compression point of 33 dBm (2 W), have a gain of 16 dB and are capable of operating in the frequency range of between 41 GHz and 46 GHz. Suitable devices include TriQuint's semiconductor device TGA4046-EPU.

The signals S₁-S₁₆ are fed to the conducting radiating patches 154 on eighth layer 116 in a way that causes adjacent patches to emit radiation orthogonally to each other.

It may be desirable to have independent control of the microwave power supplied to each of the radiating patches so that the overall field can be focussed (steered) in a way to adjust for variations in the impedance of the region of tissue being treated. This independent control is effected by the signal control devices 152 mounted in the fifth layer 114. As shown in FIG. 7, each signal control device comprises a front forward directional coupler 156, a phase shifter (e.g. a PIN diode or a varactor diode) 158, a forward power directional coupler 160 and a reflected power directional coupler 162. The couplers 156, 160, 162 are arranged to detect the power travelling either in the forward direction through the device or in the opposite direction where a signal has been reflected from the tissue back towards the source. The signals are fed to the microprocessor 124 via a phase and/or magnitude detector circuit 155. The detector may take the form of a heterodyne receiver where it is desirable to measure both phase and magnitude information, or it may take the form of a homodyne receiver where only magnitude information is required. A simple diode detector may also be used where it is only necessary to detect and process magnitude information. On the basis of these signals, the microprocessor (and/or DSP) can calculate any impedance mismatch that may occur and adjust for it by sending the necessary control signals to the phase shifter 158.

In other words, the directional couplers 156,160,162, and the microwave detectors or receivers (e.g. of the heterodyne, homodyne or diode type) measure the phase and/or magnitude of the forward and reflected power signals. These signals are then used to control the energy delivery profile via the phase shifter 158. Whilst a phase shifter (e.g. PIN or varactor diode) changes only the phase of the signals, a matching filter, which may change both magnitude and phase can be used.

FIG. 6 shows a representation of the fifth, sixth, seventh and eighth stacked layers 112,113,114,116 respectively of FIG. 5, showing the feed connections between the components on those layers. In practice, the components of adjacent layers will be on top of one another; for clarity, FIG. 6 shows the layers in a concentric arrangement.

The arrangement shown in FIG. 6 is for a microwave energy source to be split between sixteen conducting patches. The fifth layer 112 has fifteen one-to-two power splitters 148 (SP₁-SP₁₅) mounted thereon in a cascading array to split the original microwave energy source into sixteen separate sources or signals. Thus, the original source is split into two by one first generation splitter SP₁; each of the two resulting sources being further split into two by second generation splitters SP₂, SP₃; each of those four resulting sources being further split into two by third generation splitters SP₄-SP₇; finally, each of those eight resulting sources being further split into two by fourth generation splitters SP₈-SP₁₅. Each output from the fourth generation splitters SP₈-SP₁₅ is fed to a respective one of sixteen amplifiers 150 (Amp₁-Amp₁₆) in the sixth layer 113. The amplifier outputs are then fed via respective signal control devices 152 (C₁-C₁₆) in the seventh layer 114 to a respective radiating patch 154 (P₁-P₁₆) in the eighth layer 116. The patches 154 are square, which means that the emitted field comes mostly from the two opposite edges. In FIG. 6, the radiating edges 155 are indicated by thick lines, whereas the non-radiating edges 153 are indicated by thin lines. The feed lines are connected to the patches 154 to ensure that the radiating edges 155 of adjacent patches are orthogonal to one another. This may maximise the field uniformity produced over the area of the radiating antenna array, which, in turn, maximises the chances of being able to produce uniform tissue effect over the area of the antenna array.

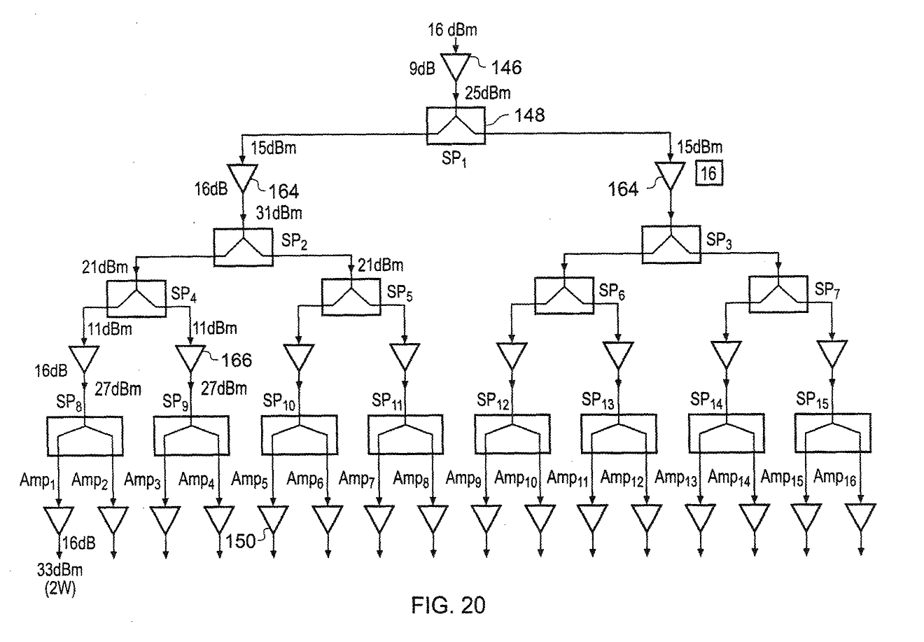

In practice, feed line losses may need to be taken into account in the structure shown in FIG. 6. In particular, buffer or booster amplifiers may need to be included to maintain a suitable signal level through the device. Each power splitter 148 typically has a loss of 3 dB associated with it. At 45 GHz, a feed line loss between components of up to 7 dB is possible, which would lead to an overall loss of up to 10 dB along each path (microstrip line) of the power splitter cascade. This loss can be compensated for by placing a buffer amplifier before every or every other power splitter. The actual configuration depends on the power budget calculated for a device. An example of a power budget is described with respect to FIG. 20 below.

One important feature of the present invention is the means by which power is transferred from the energy source to the radiating elements. Each patch antenna contained within the patch array has to be fed with microwave energy. Generally speaking, there are two main feed structures: parallel feed and series feed.

The parallel feed has a single input port and multiple feed lines are connected in parallel to constitute the output ports. Each of the feed lines is terminated at an individual radiating element (or patch).

The series feed consists of a continuous transmission line from which small portions of energy are progressively coupled into individual elements disposed along the line by various means, including proximity coupling, direct coupling, probe coupling, or aperture coupling. The series feed constitutes a travelling wave array if the feed line is terminated in a matched load, or a resonant array if it is terminated in an open circuit or a short circuit.

One example of a series feed is a radiating transmission line or a ‘leaky feeder’, which may consist of a transmission line that carries a travelling wave with a set of radiating elements. Each element would radiate only a small fraction of the total power, and by adjusting the size of each element progressively along the line, a near uniform power intensity versus length would be achievable. In this instance, the elements are not in phase as required for a conventional far field antenna, but this should not be of importance in this application. In this arrangement, the impedance of each radiating element must be lower than the characteristic impedance of the transmission line, for example, the impedance of the radiating elements may be 12.5Ω when the transmission line feed impedance is 50Ω, otherwise too much power will be radiated by the first couple of radiating patches and the return loss at the input will be poor (mismatch condition). It may be preferable to vary the size of the radiating patches in order to maintain uniform power along the radiating structure. Possible materials that could be used for constructing the patch antenna array are NovaClad from Sheldahl, thin copper clad PTFE/glass from Taconic, or R/Flex liquid crystalline polymer circuit material from Rogers Corporation.

Both parallel and series feeds can be realised as either coplanar waveguide with the radiating elements or in a separate transmission line layer. Feed lines laid in the same plane as the patches will radiate and could interfere with the radiation emitted by the radiating patches—this may not be a problem if the feed lines are controlled transmission lines and radiation is forced out of the radiating patches. This problem may also be overcome by suspending the radiating patches above the feed lines, for example, a coplanar waveguide fed suspended patch antenna array may be fabricated.

When designing the feed structure for the patch array, consideration should also be given to conductor and dielectric losses (which are typically a function of frequency of operation) and spurious radiation due to discontinuities such as bends, junctions and transitions. These losses constitute the overall insertion loss of the feed and are an important determining factor when considering the maximum possible power that can be delivered to each radiating patch. In the design of these feed structures, realisable high characteristic impedance feed lines, for example 200Ω, may be used to minimise feed-line degradation. The number of divider stages should be kept to a minimum to reduce insertion loss or feed line loss and optimisation complexity.

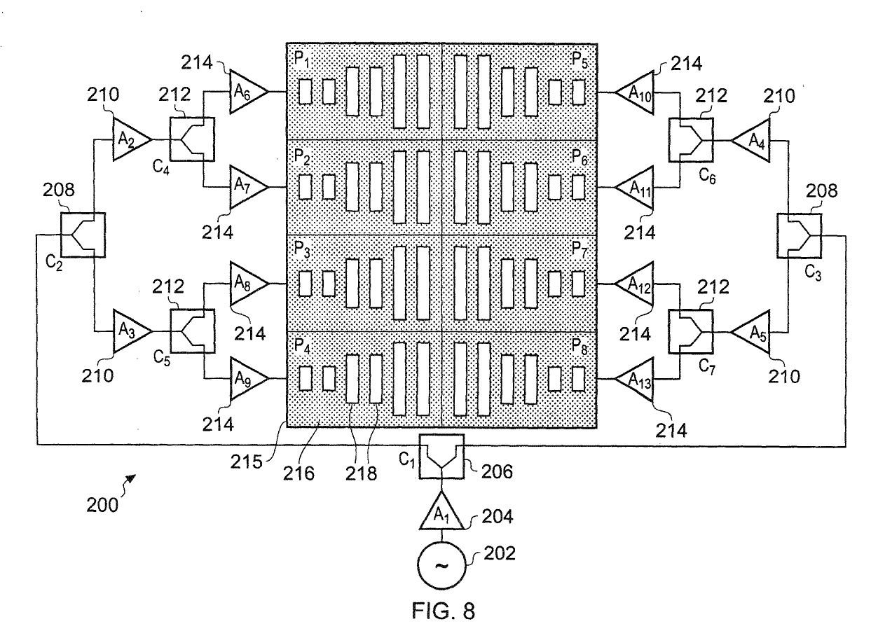

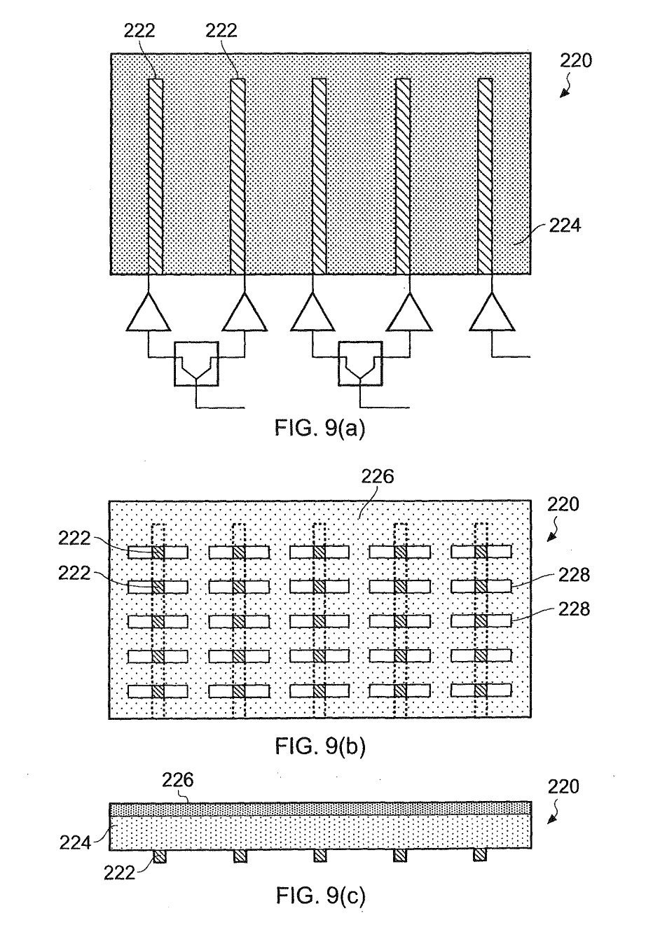

FIGS. 8 and 9(a), 9(b) and 9(c) show skin treatment devices that are based on a slotted antenna arrangement. In FIG. 8 the slots increase in width along the feed line. This is a proven method of ensuring that the same amount of microwave energy is emitted from each slot, and provides a viable application for sub-dermal treatment or skin rejuvenation or resurfacing. The structure comprises an array of slots formed in (e.g. cut into) the ground plane. Microstrip lines are fabricated onto a substrate layer whereby the lines (not shown in FIG. 8) go across the slots. An advantage of this structure is that it is relatively easy to fabricate feed lines on top of the substrate. Electromagnetic field simulation tools are used to optimise the structure in terms of slot spacing and slot size since the relationship between slot size (length) and distance from the microwave energy feed (source) to the slot is not usually linear. It has been discovered that the length of the distal slots (those furthest away from the source) found in theory need to be increased in order to take account of the power reduction near the end of the transmission line. Empirical experiments may also be used to optimise the arrangement in an iterative manner.

The device 200 in FIG. 8 includes a source oscillator 202, which can be any of a VCO, DRO, Gunn diode, SAW device or frequency synthesiser operating at any of, or a number of, the discrete frequencies discussed herein, e.g. 14.5, 24, 31, 45, 60, 77 or 94 GHz. The output from the source oscillator 202 is fed to an array of eight slotted antennas 215 via a feed structure which includes an amplifier line-up. The output from the source oscillator 202 is firstly amplified by a primary amplifier 204 before being divided by primary and secondary 3 dB splitters 206, 208 into four signals. Each of these signals is amplified by a secondary amplifier 210 before being divided into two by a tertiary 3 dB splitter 212. Each of the eight resulting signals is amplified again by a tertiary amplifier 214 before being fed to its respective slotted antenna 215.

As shown in FIG. 8, each antenna 215 has a grounded conductive layer 216 with slots 218 formed therein. The slots 218 increase in width along the length of the antenna 215 so that the energy emitted from each slot is the same, and the field from the ensemble of slots is uniform. The dimensions of the slots may be determined by using electromagnetic field simulations.

The structure of the slotted antennas can be further understood with reference to the alternative arrangement shown in FIGS. 9(a), 9(b) and 9(c), where various views of an alternative slotted antenna structure 220 are given. FIG. 9(a) shows a top view, where a plurality of microstrip feed lines 222 are fabricated on a dielectric substrate 224. Each line is fed with a microwave power signal from an amplifier line-up as discussed above.

FIG. 9(b) shows the bottom (skin-facing) surface of the device. Here a grounded conductive layer 226 is fabricated on the dielectric substrate 224. Slots 228 (shown with equal width for convenience) are formed in the grounded conductive layer 226 and dielectric substrate layer 224 to expose parts of the microstrip feed lines 222. The structure is designed so that the slots 228 act as radiating elements. The size of the slots is chosen depending on the wavelength of the radiation at the frequency of operation. Actual values may be obtained from electromagnetic field simulations. The thickness of the dielectric substrate 224 is chosen to be much less that 1 wavelength. FIG. 9(c) shows a side view of the antenna 220.

The microstrip lines 222 are preferably set up to enable the maximum E field or the maximum H field to be radiated through the slots and into the tissue. The lengths of the slots is therefore around half a wavelength. When high microwave frequencies (e.g. 31, 45, 60, 77 or 94 GHz) are used, the slots can be positioned in close proximity to one another, thus providing the required conditions for the generation of uniform energy over the entire surface of the applicator, with limited depth of penetration by microwave radiation.

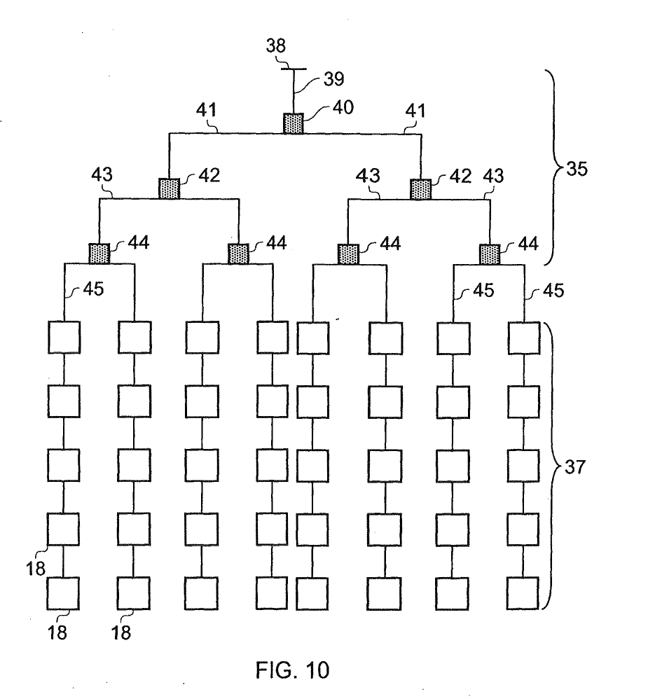

FIG. 10 shows a specific example of a feed structure that can be used in the current invention; a corporate (parallel) feed 35 may be used to feed a plurality of series connected radiating patches 37. A detailed description of this arrangement is given below. For very large arrays, the length of the feed lines running to each of the radiating elements may be prohibitively long, which will result in an unacceptably high insertion loss. For example, it is possible that at 45 GHz, the insertion loss may be several dB for a length of only a few centimetres. In designing an effective symmetrical corporate fed array, the following steps must be taken:

1) Ensure that radiating patches are matched to feed lines through appropriate dimensioning of coupling structures, or by using quarter-wave transformers.

2) Ensure that each pair of feed lines from neighbouring elements is connected to a T-junction, which is matched to the input line, if necessary through a quarter wave transformer.

3) Repeat until the last stage is reached where the feed line is connected to the feed point of the array.

In the corporate feed arrangement shown in FIG. 10, the radiating patches 18 have an input impedance of 200Ω at the edge and are connected to feed lines 45 with a characteristic impedance of 200Ω. The feed lines 45 from neighbouring elements are joined using a T-junction and transformed back to a single supply line 43 (with a characteristic impedance of 200Ω) using a 140Ω quarter wave transformer 44. A transformer that has a length corresponding to an odd multiple of a quarter of the wavelength at the frequency of interest (i.e. its length is (2n−1)λ_{L}/4, where λ_{L} is the loaded wavelength and n is an integer) will also perform the same transformation if it is assumed that the line is lossless. At short wavelengths, it may be practically necessary to use a line having a length greater than one quarter wavelength, i.e. having a length equal to an odd multiple of quarter wavelengths. The properties of the dielectric material must be stable in order to ensure that the transmission line acts as an impedance transformer. This feature is of particular importance when transformers longer than λ/4 are used, i.e. ¾λ or 5/4λ, etc., since the desired quarter electrical wavelength will otherwise be modified to an electrical length that is undesirable, for example, in the worst instance, it could end up as a multiple of a half the electrical wavelength and provide no transformation whatsoever. In the next step, neighbouring pairs of supply lines are then joined at another T-junction where they are similarly transformed through a 140Ω quarter wave transformer 42 back to a further single supply line 41 (the characteristic impedance is 200Ω). This process is repeated so that the pair of further supply lines 41 are joined at a last T-junction. A final transformation uses a 71 Ω quarter wave transformer 40 to match the parallel combination of the two 200Ω lines (i.e. 100Ω) with an input line 39 (characteristic impedance=50Ω) from energy source 38 used to feed the overall array. The impedance matching is calculated using the formula, i.e. Zₜᵣₐₙₛ=√(ZᵢₙZₒᵤₜ), which, in this case, corresponds to √(50×100)=71Ω for the last junction.

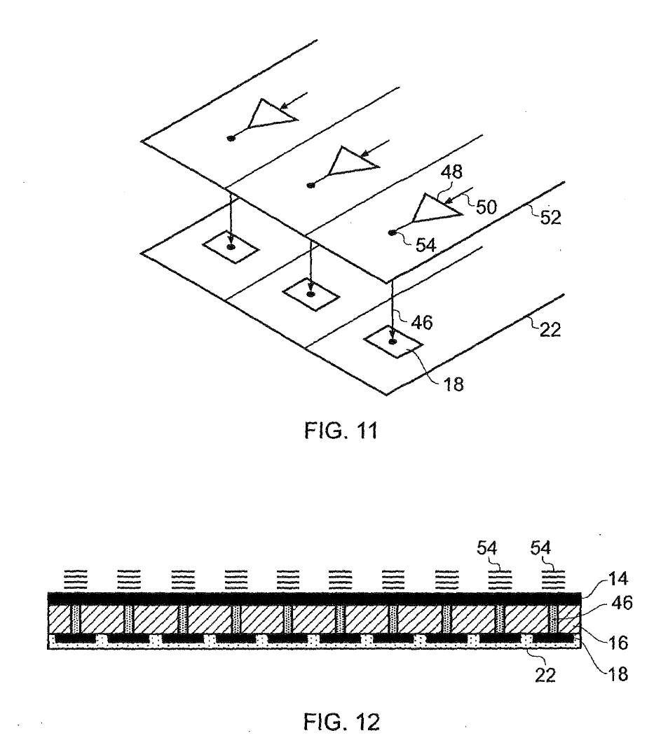

FIGS. 11 and 12 illustrate another specific example of a feed structure that can be used in the present invention. Here an array of patches (numbering 8, 16, 32, 64, 128, etc. depending upon the size of treatment zone) is arranged so that each patch is fed by a single MMIC amplifier. FIG. 11 shows a perspective view of this arrangement, where a plurality of power amplifiers 48 are mounted on an upper layer 52 of the device. They are arranged to receive an input signal 50 from the stable frequency energy source (not shown). Their output signal is fed e.g. using a low loss transmission line to a coaxial connector 54 (e.g. an SMA connector) whose outer conductor is connected to the grounded conducting plane (not shown) and whose inner conductor 46 is a conducting radiating patch 18 (shown here on the superstrate 22). FIG. 12 shows a cross-sectional view of this connection in more detail. Each patch 18 has a coaxial connector 54 associated with it. The outer conductor of each coaxial connector 54 terminates at the conducting ground plane 14, whereas the inner conductor 46 penetrates the plane and passes through the substrate layer 16 to its respective patch 18. By locating the amplifiers on a separate layer from the radiating elements, the corporate feed network (transmission lines or the like) can likewise be etched onto a layer other than the layer that contains the radiating patches. This can minimise any interference between the feed structure and the radiating patches. With good design practice, it is possible to fabricate the feed lines on the same side as the radiating patches even when the whole structure is in contact with tissue, but it is preferable to keep the feed lines and the patches separate. The idea of providing a space between the radiating patches and the tissue is also desirable in embodiments where the feed lines are on the same side as the radiating patch antennas. In order to compensate for feed line losses that can occur when high frequency, e.g. SHF or EHF, radiation is used, buffer or booster amplifiers are included in the feed structure, e.g. between one or more of the power splitters in the fifth layer 112 shown in FIG. 5.

TriQuint Semiconductor manufacture devices that are suitable for use as the power amplifiers in the present invention. In particular, TriQuint's TGA4505-EPU parts can be used for operation over a bandwidth of between 27 GHz and 31 GHz, and produce power levels of up to 36 dBm (4 W) in compression (1 dB compression point) and provide a gain of 23 dB. The dimensions of these MMIC chips are around 2.8 mm×2.2 mm×0.1 mm. If one device is used to feed four patches and the length of feed lines is kept very short, power levels of up to 1 W may be radiated from each patch. More recently, amplifiers that work up to 45 GHz (e.g. TriQuint's TGA4046-EPU) have become available; these parts can provide up to 2 W of power. Due to recent developments and interest in mm wave technologies and terahertz systems, energy at high microwave and mm wave frequencies with associated small depths of penetration is becoming more readily available, and so it will be possible to produce high localised energy densities inside the tissue using these devices.

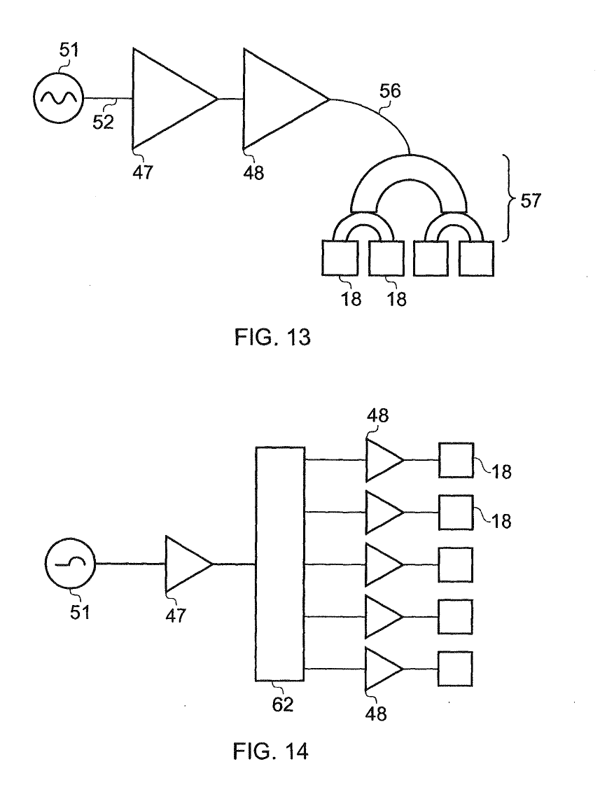

FIG. 13 illustrates schematically an amplifier line-up for a 4 W generator that may be used in an embodiment of the present invention. The line-up comprises a suitable frequency source 51, which may be a closed loop phased locked dielectric resonator oscillator (DRO) using a single or a plurality of temperature compensated crystal oscillator references, or a temperature compensated open loop DRO. Other frequency sources, such as a Gunn diode oscillator or a voltage controlled oscillator (VCO) can be used; the choice of oscillator depends on the frequency being used. The output 52 of the frequency source represents a stable frequency signal which is fed into a pre-amplifier 47 (here TriQuint's TGA4902-EPU-SM device), which has a 1 dB compression point of 25 dBm. In general, monolithic microwave integrated circuits (MMICs) are suitable for use as pre-amplifiers. For frequencies up to about 20 GHz, gallium arsenide (GaAs) based MMICs are preferred. For frequencies beyond this and up to 100 GHz, high electron mobility transistor (HEMT) based MMICs or metamorphic HEMTs can be used. For example, suitable MMICs for 31 GHz and 45 GHz operation are TriQuint's TGA4902-EPU-SM and TGA4042-EPU parts respectively. The output of the pre-amplifier is fed into the power amplifier 48 (here TriQuint's TGA4505-EPU MMIC device). For frequencies up to about 20 GHz, gallium arsenide (GaAs) or gallium nitride (GaN) transistors or MMIC devices are suitable for use as power amplifiers. For frequencies beyond this and up to 100 GHz, it may be preferable to use high electron mobility transistor (HEMT) based devices. Examples of suitable power MMICs for 31 GHz and 45 GHz operation are TriQuint's TGA4505-EPU and TGA4046-EPU parts respectively.

Typically, the power level from the frequency source is in the range of −10 dBm to +15 dBm, and depends on the type of source oscillator used, which is itself governed by the desired frequency of operation. For example, a typical DRO oscillator may produce power in the range −5 dBm to +5 dBm. If the power level output provided by the frequency source 51 is −5 dBm and the gain of the pre-amplifier 47 is about 18 dB, the power level input to the power amplifier 48 is 13 dBm. The gain of the power amplifier 48 is about 23 dB, so the power level at the output 56 is 36 dBm (4 W). An impedance matched corporate feed structure 57 (see description of FIG. 10 above) splits the output 56 into individual microwave power sources for exciting the four radiating patches 18.

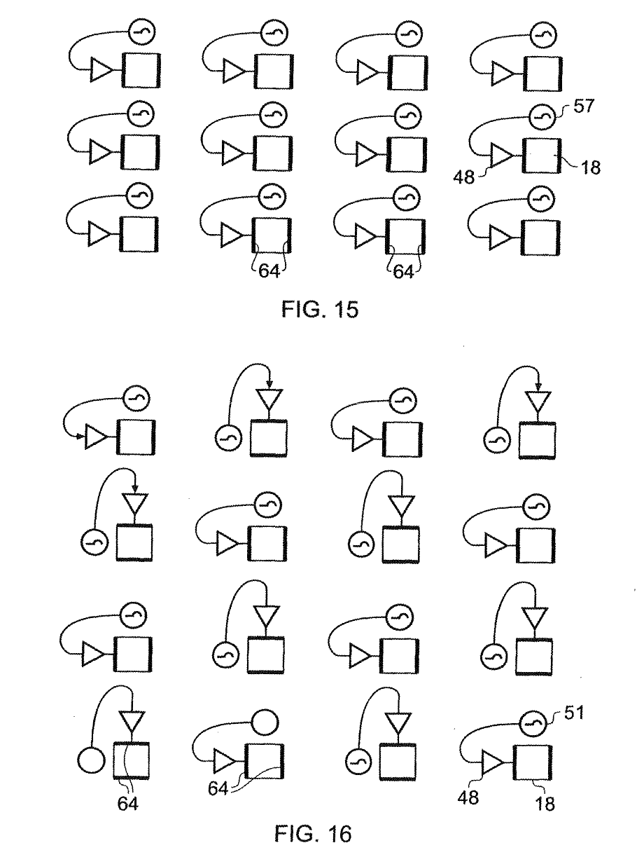

FIG. 13 shows an arrangement where a single source oscillator 51 is followed by single pre-amplifier 47 and a single power amplifier 48 feeding a corporate distribution network 57. Other distribution arrangements that use corporate feed networks are also possible. FIG. 14 shows an arrangement where a single source oscillator 51 and a single pre-amplifier 47 are followed by a power splitter 62, which provides an input to a plurality of power amplifiers 48, each of which feed a single radiating patch 18. FIG. 15 shows an arrangement where a separate source oscillator 51 and power amplifier 48 are provided for each radiating patch.

In FIG. 15, the power input to each patch is arranged so that the same (i.e. parallel) edges 64 on each patch radiate. However, to improve further the uniformity of the radiated field, it is desirable to arrange the input feeds so that the radiating edges 64 on adjacent patches are orthogonal to one another. FIG. 16 shows a separate source oscillator 51 and power amplifier 48 for feeding each radiating patch 18 where the feeds are provided on alternating edges of adjacent patches to cause orthogonal edges 64 to radiate and thereby ensure a more uniform field distribution, which can lead to a uniform tissue effect. In other words, the patch array is set-up in such a manner that the two edges of the patches that are dominant in producing the fringing fields are alternated between adjacent patches. Thus, in FIG. 16, adjacent patches are fed orthogonally and each feed line is designed such that the output fields are in phase to produce a uniform field over the surface of the skin.

As explained above, the device is optimised e.g. using electromagnetic field modelling to ensure the antenna structure is impedance matched to the characteristics of the biological tissue and that the fields inside the skin tissue are uniform. The feed structure can also be modelled using microwave simulation tools such as Ansoft HFSS, Flomerics Microstripes or CST Microwave Studio®.

Electromagnetic field modelling helps in the determination of the position of the feed lines with respect to the patch. For example, the position of the feed line determines the feed impedance or the impedance seen by the radiating patch. In the instance of the co-axially fed patch, where a wire or pin is connected to the back of the patch and the wire or pin is inserted through the substrate or dielectric layer, the position of the pin with respect to the area of the patch determines the feed impedance. It is important to ensure that the feed line is matched to the antenna in order to minimise the level of reflected power. The position of the feed onto the patch also determines the two edges of the patch that radiates. Thus, in the instance whereby it is desirable that adjacent patches radiate orthogonal fields, the position of the feed line with respect to the area of the patch determines this pattern.

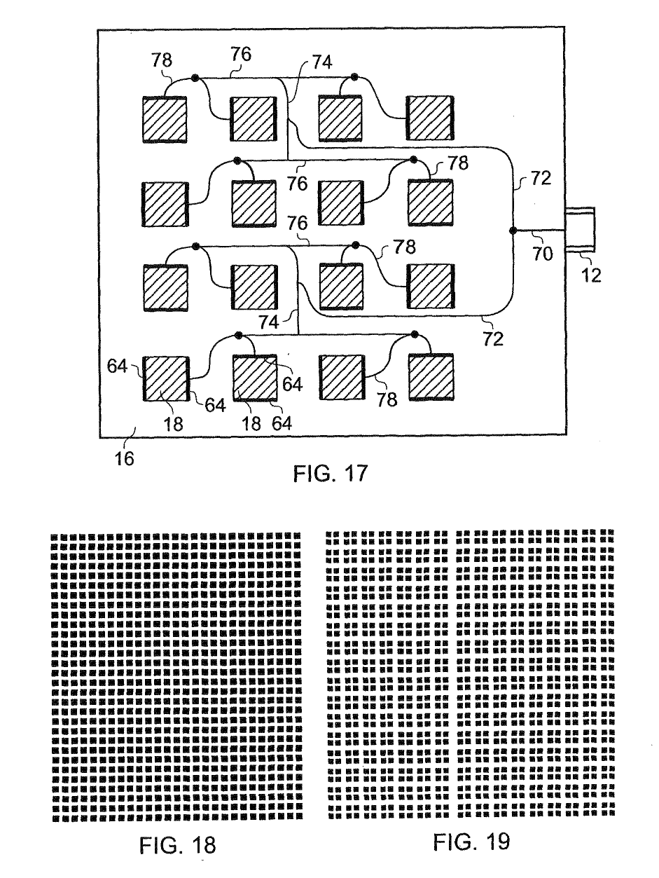

FIG. 17 shows a practical embodiment of the arrangement shown in FIG. 16. Sixteen conducting patches 18 are mounted on a substrate layer 16 in a 4×4 array. Microwave energy is delivered from an energy source feed connector 12, from where it is delivered to each patch via a corporate feed structure comprising a plurality of transmission lines 70,72,74,76,78. Primary feed line 70 from feed connector 12 splits into two secondary feed lines 72, each of which splits into two tertiary feed lines 74, each of which split into two quaternary feed lines 76, each of which split into two quinary feed lines 78 (giving sixteen in total), each of which is connected to a radiating patch 18. The transmission lines are arranged so that adjacent patches are fed (i.e. have their respective quinary feed line connected to) at edges 64 that are orthogonal to one another. The feed structure is also impedance matched as described above.

As mentioned above, a superstrate layer, e.g. a dielectric cover, located between the radiating patches and the surface of the skin can be used to augment uniformity of tissue effect by dispersing the fields and provide a disposable element between the e.g. metallic radiating patch array and the human tissue. This layer may also provide a degree of thermal isolation between the radiating patch array and the surface of the skin. It is desirable for said cover to be a disposable item rather than having the complete patch antenna array as a disposable item for cost reasons. The superstrate is therefore removable from the rest of the device, to enable it to be easily fitted by non-trained medical personnel. For example, it may be snap-fitted into place. It is desirable to have a close fit to prevent air gaps from causing an impedance mismatch condition. A locking mechanism, e.g. clips around the edge of the device may be used to fix the superstrate in place during use.

An alternative to the above would be to provide a conformal coating to the patch antenna array applicator using a biocompatible material such as Parylene C or Teflon®. In this instance the complete device would form the disposable item. It should be noted that the dielectric cover will affect the performance of the patch antenna array applicator to such an extent that it must be taken into account when designing the patch antenna array. Generally speaking, a dielectric cover will cause the resonant frequency to be lowered. Therefore, the patches should be designed to resonate at a slightly higher frequency than the operating frequency of choice. When the patch array is covered with said dielectric cover the properties that will change include: the effective dielectric constant of the substrate material, the losses, the Q-factor and the directive gain. Given the unusual environment that the patch array will be operating in, the Q-factor and the directive gain should not need to be considered in the same manner as they would if the patch array was to be operating in a conventional environment, i.e. as a part of a RADAR system, or in a line of sight communications link. The change in the effective dielectric constant due to the cover will present the greatest change, and the amount of change is governed by the thickness and the relative permittivity of the substrate material. The presence of the cover layer also produces changes in the radiation pattern produced by the antenna array.

It is also worthwhile noting that the superstrate layer will help ensure an even field distribution, or uniform tissue effect. The correct choice of dielectric constant and loss factor (1/Q or tan δ) may enable enhanced field uniformity. It may be preferable to form the superstrate layer from a plurality of materials, with different dielectric properties to enable the wave produced by individual radiating antennas to be slowed down by different amounts. The materials may be varied over the surface area and the thickness (the depth) of the various materials may be varied. This feature may enhance the field uniformity produced over the surface of the applicator (the antenna) array.

As mentioned above, the skin treatment device of the present invention receives its power from an energy source. The energy source includes a source oscillator, e.g. a voltage controlled oscillator (VCO) or a dielectric resonator oscillator (DRO). For frequencies above 15 GHz, a DRO is preferred; VCOs generally use LC tuned circuits, which are typically limited to frequencies of up to 15 GHz. Other devices that could be used include: Gunn diode oscillators and Surface Acoustic Wave (SAW) oscillators. It may be preferable to use a closed loop phased locked DRO, or a temperature compensated open loop DRO, in order to maintain a stable single operating frequency. It may also be preferable to drive individual radiating patches or groups of radiating patches with source oscillators operating at different frequencies, i.e. a plurality of source oscillators may be used, where each individual oscillator outputs a different frequency to feed a group of radiating patches. It may be preferable to use a frequency synthesiser to produce a plurality of fixed (stable) frequencies. One embodiment described above is based on an operating frequency of 14.5 GHz, where semiconductor power devices are readily available. The size (surface area that may be treated by the device) can vary between less than 0.5 cm² and greater than 10 cm². FIG. 18 shows a scale view of a patch antenna array with a treatment surface area of about 8 cm×9 cm where the size and separation of each patch is calculated to be suitable for radiating an electromagnetic field at 14.5 GHz into wet skin. Other embodiments can be designed to operate at higher frequencies (e.g. 24 GHz, 31 GHz, 45 GHz, 60 GHz, 77 GHz, 94 GHz or higher) which offer the advantage of enabling more dense arrays to be formed and a smaller depth of penetration of radiation to be achieved. At higher frequencies (e.g. 45 GHz or above), the energy sources (e.g. power amplifiers) may be connected directly to the radiating elements (radiating patches) to further reduce or minimise feed line loss. At higher frequencies, lower penetration depths are achievable. FIG. 19 shows a scale view of a patch antenna array with a treatment surface area of about 6.5 cm×6.5 cm where the size and separation of each patch is calculated to be suitable for radiating an electromagnetic field at 31 GHz into wet skin. Each patch is generally separated from its adjacent neighbours by a distance of around λ_{L}/2, where λ_{L} is the loaded wavelength. The separation distance is therefore reduced as frequency increases. In practice, the size of the gaps will be calculated precisely using a computer simulation tool to optimise the uniformity of the radiated fields and the tissue effects.

FIG. 20 illustrates another view of the power splitter network of the fifth layer 112. The network in FIG. 20 has buffer amplifiers 164,166 located at selected positions between the power splitters to ensure that the signal amplitude remains at a suitable level (despite feed line losses etc.) to drive the amplifiers 150 in the sixth layer 113. The power budget for the feed structure in FIG. 20 is explained below.

Before the input to the network of power splitters 148, the power amplifier 146 (having a gain of 9 dB and a 1 dB compressed power rating of 28 dBm) increases the power from preamplifier 144 from 16 dBm to 25 dBm. This level is then split into two equal parts using a 3 dB splitter SP₁, and a feed line with an estimated insertion loss of 7 dB, this gives an input power of 15 dBm at the input to each of the first buffer amplifiers 164, which have a gain of 16 dB. The first buffer amplifiers 164 therefore produce an output power of 31 dBm. TGA4046-EPU components from TriQuint can be used as the first buffer amplifiers. The outputs from the first buffer amplifiers 164 are split using 3 dB splitters SP₂ and SP₃, and with feed line losses taken into account, provide four balanced outputs at a power level of 21 dBm. These output powers are further split using 3 dB splitters SP₄-SP₇, to give eight balanced outputs of 11 dBm. These output powers are then amplified with second buffer amplifiers 166 which have a gain of 16 dB (e.g. TGA4046-EPU devices from TriQuint semiconductor). The output power from each buffer amplifier 166 is therefore 27 dBm, and each of these outputs is used to feed a respective one of eight power splitters SP₈-SP₁₅.

With feed line losses taken into account, the output power from each of the two split parts of each of the eight splitters SP₈-SP₁₅ is 17 dBm. These outputs are fed into the input ports of the sixteen power amplifiers 150 (Amp₁-Amp₁₆) in the seventh layer 113. Their outputs are connected directly to the radiating patches (not shown). The devices used here are TriQuint's TGA4046-EPU components with a gain of 16 dB and compressed power of 33 dBm. Thus the arrangement is therefore capable of driving 33 dBm (2 W) into each of the sixteen radiating patches to produce a range of desirable tissue effects.

If desired, additional buffer amplifiers could be included between the group of two power splitters SP₂,SP₃ and the four power splitters SP₄-SP₇. The buffer amplifiers may then have a lower gain.

A further implementation of applicators or antenna arrays that may be used when working at the higher end of the frequency range, e.g. 45 GHz, 60 GHz or higher is discussed below. At these frequencies, coplanar waveguide fed suspended patch antenna array structures may be preferred. These alternative structures may comprise of coplanar waveguide feed lines, appropriate feeding posts and square or rectangular radiating patches. Coplanar waveguide structures have the ground plane and the signal line on the same surface, hence when the radiating patch is supported with a feeding post, the ground plane of the coplanar waveguide structure can be used as the ground plane for the radiating patch, i.e. the air between the underside of the radiating patch and the ground plane forms the dielectric substrate. The coplanar waveguide structure can be mounted on a dielectric material or substrate with a high dielectric constant and the radiating patch antenna sits on a layer of air. Because the radiating patch is supported with metal posts (or metallised plastic supports) in air, there are no dielectric losses, thus the performance of the radiating patch antenna may be better than that of a conventional microstrip based antenna structure where a dielectric material is sandwiched between the radiating patch antenna and the ground plane.

The structure described below is similar to the co-axial feed arrangement discussed earlier, where a wire or pin is connected to the radiating patch and said pin is fed through the dielectric substrate material to enable an electrical connection to be made using, for example, a direct connection method where a microwave connector is connected directly to the radiating patch.

The feed post for the proposed coplanar waveguide antenna structure acts simultaneously as a signal line and a mechanical support for the radiating patch antenna. It is possible to select the desired input impedance for the patch antenna by carefully selecting the location of the feed post. This impedance is preferably chosen such that the feed line can be directly matched to the radiating patch antenna without the need to use a quarter-wave impedance transformer.

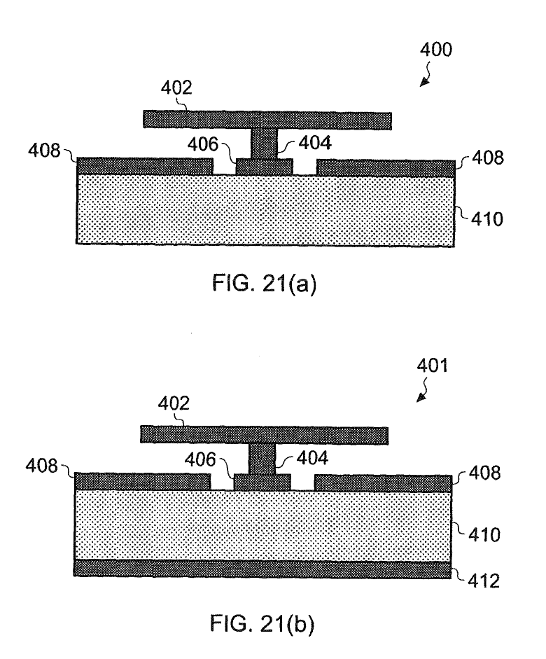

FIG. 21(a) shows a coplanar waveguide structure 400 in which a single radiating patch antenna 402 is fed via a feed post 404. The coplanar waveguide is formed from a signal conductor 406 separated from a pair of ground planes 408, all on the same side and attached to the first surface of a dielectric material 410. In this arrangement, much less field enters the dielectric 410 when compared with a microstrip structure in which the signal conductor is connected to the first surface of the dielectric and the ground plane is connected to the second surface of said dielectric.

The dielectric thickness may be great enough to ensure that the electromagnetic fields are substantially reduced by the time they get to the outside world, i.e. by the time they reach the second surface of the dielectric material and propagate into air.

FIG. 21(b) shows a variant 401 of the structure in FIG. 21(a). In this arrangement, the second surface of the dielectric material is fully covered with a conductor 412 that forms a further ground plane. This structure is known as a ground-plane coplanar waveguide or a grounded coplanar waveguide structure. The advantage of using these coplanar waveguide feed structures over conventional microstrip feed structures is that the coplanar structure can operate up to and beyond 100 GHz frequencies due to the fact that connecting the coplanar waveguide does not entail parasitic discontinuities in the ground plane as is the case for microstrip structures; the effect of the parasitic elements become more prevalent as the frequency of operation is increased.

FIGS. 21(a) and 21(b) show the radiating patch antenna electrically and physically connected to the coplanar waveguide feed structure using a single feeding post. A plurality of posts may be used to support the radiating patch. Where posts are connected between the radiating patch and the ground plane, the material used for the posts is desirably a low loss dielectric material. Alternatively, quarter wave stubs may be used as posts between the ground plane and the radiating patch antenna and the posts maybe positioned such that they are electrically transparent to the microwave signal. The length of the posts is typically less than 1 mm, e.g. 0.3 mm, so it is practical to use micromachining technology to fabricate the structure.

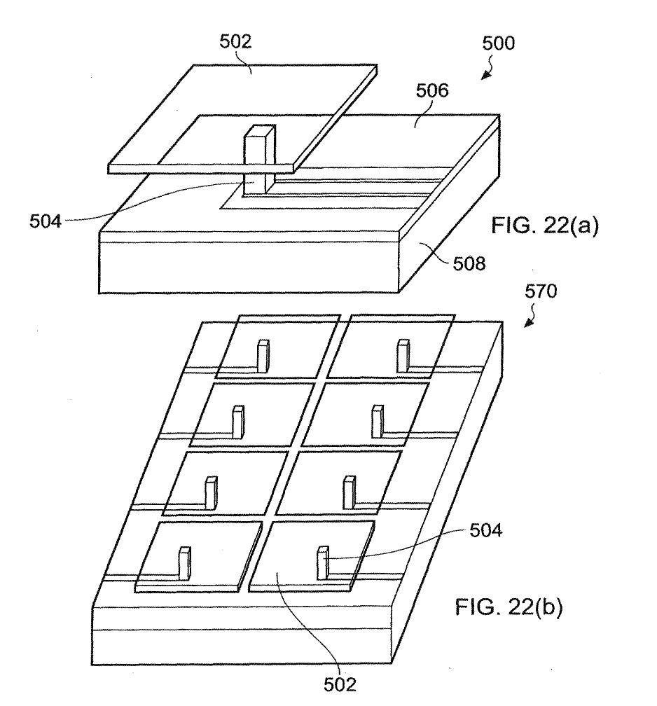

FIG. 22(a) shows an arrangement 500 for a single radiating patch antenna 502 suspended above a coplanar waveguide feed structure using a feeding post 504. The arrangement 500 uses a conventional coplanar waveguide structure where the ground plane 506 exists only on the first surface of the dielectric material 508.

FIG. 22(b) shows an array 510 of eight radiating patch antennas 502, each fed using a separate feed post 504 with one end connected to the radiating patch antenna and the other end connected to the coplanar waveguide structure.

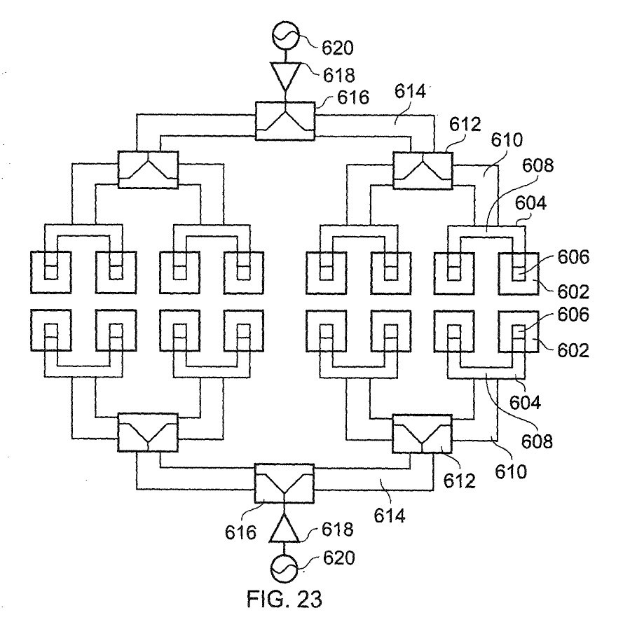

FIG. 23 shows another embodiment of this aspect of the invention in which an array of sixteen radiating patch antennas 602 are each connected to the signal line 604 of coplanar waveguide structures using feeding posts 606. In FIG. 23, the radiating patch antennas 602 are separated into adjacent pairs, each pair being joined together using a single coplanar waveguide feed line respectively. In this embodiment, the input impedance of each radiating patch antenna 602 is 100Ω. Thus, if signal lines 604 have a characteristic impedance of 100Ω then the centre point 608 of the lines, where the energy is fed into the structure, is 50Ω, i.e. the combination of two 100Ω impedances connected in parallel. This arrangement may be advantageous in that it is not necessary to use a quarter-wave transformer to transform the input impedance of the radiating patch antennas to the output impedance of the source or generator, which is normally 50Ω.

The centre point 608 of each signal line 604 is connected to one end of a planar microstrip line 610. The characteristic impedance of the microstrip line 610 is 50Ω. The other end of the microstrip lines 610 are grouped into pairs, each pair of microstrip lines being connected to the output port of a power splitters 612. The power splitters 612 are 3 dB power splitters with the input port and the two output ports designed to accept 50 Ω microstrip lines. Drop-in microstrip couplers can be used. The advantage of using 3 dB couplers is that the input power incident at the input port is split equally into two parts to enable each radiating patch antenna 602 to produce equal amounts of microwave energy. The input port of each power splitters 612 is connected to one end of a primary microstrip line 614. The characteristic impedance of the primary microstrip lines 614 is 50Ω. The other end of the primary microstrip lines 614 are grouped into pairs, each pair being connected to the output ports of primary power splitters 616. The primary power splitters 616 are 3 dB power splitters, with the input port and the two output ports designed to accept 50Ω microstrip lines. The input port of each primary power splitters 616 is connected to the output of a power amplifier 618 respectively. The power amplifiers 618 are preferably based on HEMT device technology, e.g. metamorphic HEMT technology (MHEMT), and may be a single device or an array of individual HEMT devices integrated into one unit to provide the necessary level of power required to produce the desired tissue effects. The input of each power amplifier 618 is connected to the output of a frequency source oscillator 620. The frequency source oscillators 620 may be Gunn diode oscillators or dielectric resonator oscillators, although other devices that can produce a signal at the frequency of choice may be used.

Since there are no impedance transformers in the structure, the patch antenna array can be designed with a minimal number of step changes in the lines that give rise to discontinuities that may produce unwanted radiation at the junctions or steps where the transformations take place.

The adjacent radiating patch antennas are separated by a distance equal to 0.8λ, where λ is the frequency of choice.

Where additional supporting posts are used to support the antennas, it may be preferable for the additional posts to be placed at the E-field centre of the radiating patches and be connected to the ground plane. Ideally, the additional posts do not affect the performance of the radiating antennas.

It is preferable for the lengths of the edges of the radiating patches to be a half the wavelength at the frequency of operation. The electric field under the radiating patches is maximum at the first radiating edges, zero in the middle, and maximum again at the second radiating edge. Since the electric field is zero at the middle of the radiating patch, supporting posts or electric shorting walls can be erected at these locations without disturbing the field distribution under the radiating patches. Since in the coplanar waveguide structure the ground planes are located in the vicinity of the signal lines, it is easier to guide the electric field. For microstrip transmission lines, the line impedance depends heavily on the substrate properties and it can be difficult to implement stable lines on some microwave dielectric materials at high microwave frequencies, especially those defined as being within the millimetre wave range. However, for the coplanar waveguide structure, the width of the signal line and the gap between the signal line and the ground plane can be adjusted.

The above technique may also be used at lower microwave frequencies, although the drawback is that the gap between adjacent patches will be increased and the overall field pattern produced may not be as uniform, hence the tissue effects may also be less uniform.

The feeding posts (or supports) that are used to connect the radiating patch antennas to the feed line are preferably flexible to enable the antenna array to be conformal with the surface of the tissue being treated, i.e. the skin. To implement this feature it may be desirable to make use of flexible plastic materials that can be coated or impregnated with a metallic material to form the conducting contact between the radiating antennas and the feed line within the coplanar waveguide structure. It is preferable for the thickness of said conductive coating or layer to be equal to at least five skin depths at the frequency of operation to enable the majority of the microwave energy to be transported from the feed lines to the radiating patch antennas. At the frequencies of interest for the implementation of the current invention the thickness will be around 1 μm when common conductor types are used, for example, copper (Cu) or silver (Ag); this implies that the flexibility of the non-conductive material used to form the flexible feed posts will be unimpaired. The ability to produce a structure that conforms to the surface of the skin may provide an additional feature for the current invention.

It should be noted that it may also be preferable to suspend the radiating patches that are fed using a corporate feed network, such as that described earlier in this description, or another embodiment of a planar feed network, and make use of the ability to produce an array of radiating antenna elements that can conform or adapt to the surface of the skin of the particular body part of the person being treated. In arrangements using planar structures it may not be possible to use the idea of having the ground plane for the radiating patch on the same surface of the dielectric material as the signal lines, thus co-axially fed arrangements would need to be considered, where a first pin is used to connect the signal line and a second pin (or a plurality of additional pins) are used to connect the ground plane of the radiating microstrip patch to the microstrip based feed line structure.

The suspended antenna array idea may overcome problems associated with feed line structure heating and the reduction of the energy available at the radiating patches caused by conventional planar feed line structures making direct contact with the biological treatment tissue (in this case, the surface of the skin).

Each of the suspended radiating patches may be coated with a biocompatible material or may have a block of radiating material attached thereto to ensure that the surface of the skin is not exposed to conducted heat produced by the radiating patch antennas and to assist in producing uniform tissue effects.

1-25. (canceled)

26. A device for treating skin tissue with microwave radiation, the device having:
a treating surface for locating over a region of skin to be treated;
a plurality of radiating elements on the treating surface; and
a feed structure arranged to deliver microwave energy to the radiating elements;
wherein the radiating elements are configured to emit outwardly the delivered microwave energy as an electromagnetic field at the treating surface, such that, during treatment, the emitted electromagnetic field has a uniform field distribution arranged to penetrate the region of skin to be treated to a predetermined depth.

27. A device for treating skin tissue with microwave radiation, the device having:
a treating surface for locating over a region of skin to be treated;
a plurality of radiating elements on the treating surface; and
a feed structure arranged to deliver microwave energy to the radiating elements;
wherein the feed structure includes a plurality of power sources, each power source being associated with one or more of the radiating elements, whereby the radiating elements are configured to emit outwardly the delivered microwave energy as a electromagnetic field at the treating surface, such that, during treatment, the emitted electromagnetic field has a uniform field distribution arranged to penetrate the region of skin to be treated to a predetermined depth.

28. A device according to claim 27, wherein each power source is independently controllable.

29. A device according to claim 28, wherein each power source includes a power amplifier and a monitoring unit arranged to detect the power delivered by the amplifier, and wherein the power supplied to the power amplifier is controlled on the basis of the delivered power detected by the monitoring unit.

30. A device according to claim 29, wherein the monitoring unit is arranged to detect the power reflected back to the power amplifier, and wherein the power supplied to the power amplifier is further controlled on the basis of the reflected power detected by the monitoring unit.

31. A device according to claim 29, wherein each power source includes an dynamic impedance matching unit arranged to control the power supplied to the power amplifier on the basis of information detected by the monitoring unit by matching the impedance of each radiating element to the impedance of the skin tissue to be treated.

32. A device according to claim 26, wherein the plurality of radiating elements is on an outward facing surface of a dielectric substrate layer, a grounded conductive layer is formed on a surface of the dielectric substrate layer opposite the outward facing surface, and the feed structure is arranged to deliver an alternating current to the plurality of radiating elements, the grounded conductive layer being arranged to provide a return path for the alternating current.

33. A device according to claim 32, wherein each radiating element includes a conducting patch mounted on the outward facing surface of the dielectric substrate layer.

34. A device according to claim 33, wherein each conducting patch is rectangular and configured to emit the electromagnetic field in its fundamental (TM₁₀) mode.

35. A device according to claim 32, wherein the feed structure includes a single stable microwave frequency energy source and a network of transmission lines for carrying energy from the single source to the plurality of radiating elements, the network transmission lines including a plurality of power splitters arranged to divide an output from the single source into a plurality of inputs, each input being for a respective radiating element.

36. A device according to claim 35, wherein the transmission lines are sandwiched in the dielectric substrate layer between the grounded conductive layer and the radiating elements.

37. A device according to claim 35, wherein a coaxial connection connects each radiating element and the grounded conductive layer to a transmission line.

38. A device according to claim 26, wherein the feed structure includes a coplanar waveguide and each of the plurality of radiating elements is suspended from the coplanar waveguide by a conducting feed post.

39. A device according to claim 26, wherein the feed structure is arranged to cause electromagnetic fields emitted by adjacent radiating elements to be orthogonal to one another.

40. A device according to claim 26, wherein the treating surface, radiating elements and feed structure are formed on a flexible sheet that is conformable to the region of skin to be treated.

41. A device according to claim 26, including a cover portion for locating between the treating surface and the region of skin to be treated, the cover portion being of a low loss material for dispersing the electromagnetic field from the radiating elements into the tissue.

42. A device according to claim 41, wherein the cover portion is disposable and/or biocompatible.

43. A device according to claim 26, wherein the treating surface has an area of 0.5 to 10 cm².

44. A device according to claim 26, wherein the predetermined depth of penetration is 0.05 mm to 5 mm.

45. A device according to claim 26, wherein the microwave electromagnetic field emitted by the radiating elements is arranged to heat substantially instantaneously the region of skin to be treated to a temperature of 45° C. or more.

46. A device according to claim 26, wherein the microwave energy has a frequency of more than 10 GHz.

47. Apparatus for treating skin tissue with microwave radiation, the apparatus including:
a source of microwave radiation having a stable output frequency;
a device according to any preceding claim connected to the source of microwave radiation; and
a controller arranged to control the amount of energy delivered via the microwave radiation to the tissue to be treated.

48. Apparatus according to claim 47, including a cooling device arranged to cool a treatment surface during the application of the microwave energy so that the microwave energy leaves tissue at the surface unchanged whilst affecting tissue below the treatment surface.

49. Apparatus according to claim 48, wherein the cooling device is a Peltier cooler or a coolant or freezer spray.

50. A method of treating skin tissue with microwave radiation, the method including:
covering a region of skin to be treated with a treating surface that has a plurality of radiating elements thereon;
connecting a source of microwave radiation having a stable output frequency to the radiating elements, whereby the radiating elements emit a microwave electromagnetic field which penetrates the region of skin to be treated to a predetermined depth; and
controlling the amount of energy delivered by the microwave radiation to the region of skin to be treated.
